(19) United States
(12) Patent Application Publication
(10) Pub. No.: US 2007/0219767 A1
(43) Pub. Date: Sep. 20, 2007

(54) ATOMIC COORDINATES OF ALBUMIN DRUG COMPLEXES AND METHOD OF USE OF PHARMACEUTICAL DEVELOPMENT

(76) Inventors: Daniel C. Carter, Heatherhill Circle Huntsville, AL (US); Joseph Ho, Wood Creek Drive Madison, AL (US); Zhongmin Wang, Murry Drive Madison, AL (US)

Correspondence Address: STITES & HARBISON PLLC, 1199 NORTH FAIRFAX STREET, SUITE 900, ALEXANDRIA, VA, 22314, US

(21) Appl. No.: 10/555,761

(22) PCT Filed: May 6, 2004
(86) PCT No.: PCT/US04/14046

§ 371 Date: Jul. 28, 2006

Related U.S. Application Data
(60) Provisional application No. 60/468,057, filed on May 6, 2003.

(51) Int. Cl. G06G 7/48; C07K 14/00; G01N 33/53

(52) U.S. Cl. 703/11; 435/7.1; 530/350

(57) ABSTRACT

A method is provided for evaluating drug compounds based on their binding properties to human serum albumin wherein structural information at particular albumin binding regions is entered into a computer database and assessed with regard to particular contacting binding residues located in accordance with the invention. The information obtained through the computer database is thus useful in assessing and predicting drug interactions at albumin binding sites. Further, protein fragments including one or more albumin binding sites are provided which can be used in methods of assessing and designing drugs.

### CROSS-REFERENCE TO RELATED APPLICATIONS

The present application claims the benefit of U.S. provisional application Ser. No. 60/468,057, filed May 6, 2003.

### FIELD OF THE INVENTION

The present invention relates in general to serum albumin drug binding sites and complexes at those binding sites along with methods of evaluating drug interactions at those sites through information obtained by producing a three-dimensional database of the molecular structural coordinates of the albumin binding regions. In particular, the invention relates to specific binding sites and molecular complexes in human serum albumin for which a detailed, three-dimensional database has been produced and to information learned thereby to allow the evaluation and modeling of drugs based on binding interactions at those binding sites, and to the discovery of drug binding at sites on human serum albumin that previously were not associated with drug binding, such as subdomain known as 1B or Site 1B, which now has been shown for the first time to be the major drug binding region in human serum albumin. The information obtained from computer databases produced from three-dimensional structuring of albumin binding sites can thus be used in accordance with the invention to assess and design drugs which can bind to those sites. Accordingly, the invention relates to the use of detailed structural information of albumin binding sites in situ to assess drug molecules and molecular complexes as well as to protein fragments containing one or more active binding sites which can also be used to assess drug binding activity and model drug design based on albumin binding properties. Finally, the invention also relates to the creation and use of a computer readable database of information regarding the three-dimensional molecular structural coordinates for improving the in vivo safety and efficacy of new drugs or existing pharmaceuticals, and to develop predictive capabilities in drug binding, drug displacement interactions and in silico ADME processes.

### BACKGROUND OF THE INVENTION

Human serum albumin is a major protein of the circulatory system and plays an important role in numerous physiological functions as well, including a significant contribution to colloidal oncotic blood pressure (roughly 80%) and a major role in the transport and distribution of numerous exogenous and endogenous ligands. These ligands can vary widely and include chemically diverse molecules including fatty acids, amino acids, steroids, calcium, metals such as copper and zinc, and various pharmaceutical agents. Albumin generally facilitates transfer many of these ligands across organ-circulatory interfaces such as the liver, intestines, kidneys and the brain, and studies have suggested the existence of an albumin cell surface receptor. See, e.g., Schnitzer et al., P.N.A.S. 85:6773 (1988). Serum albumin generally comprises about 50% of the total blood component by dry weight, and is also chiefly responsible for controlling the physiological pH of blood. This protein is thus intimately involved in a wide range of circulatory and metabolic functions and vitally important not only to proper circulation and blood pressure but to the interactions and effects of pharmaceutical compositions when administered to a patient in need of such administration.

Human serum albumin (or “HSA”) is a protein of about 66,500 kD and is comprised of 585 amino acids including at least 17 disulphide bridges and, as set forth above, has an outstanding ability to bind and transport a wide spectrum of ligands throughout the circulatory system including the long-chain fatty acids which are otherwise insoluble in circulating plasma. The sequences and certain details regarding specific regions in albumin have previously been set forth, e.g., in U.S. Pat. No. 5,780,594 and U.S. Pat. No. 5,948,609, both of which are incorporated herein by reference. Other articles or references of relevance with regard to human serum albumin include Carter et al., Advances in Protein Chemistry, 45:153-203 (1994); Peters, Jr., “All About Albumin”, Academic Press (1995); Camerman et al., Can J. Chem., 54:1309-1316 (1976); Lau et al., J. Biol. Chem., 249:5878-5884 (1974); Callan et al., Res. Commun. Chem. Pathol. Pharmacol., 5:459-472 (1973); and Nieboer et al., Br. J. Ind. Med., 41:5663 (1984); and all of these references are incorporated by reference as well.

HSA is thus one of the major circulatory proteins, and because of its abundance in the circulatory system, it is one of the prime determinants of the safety and efficacy of many pharmaceuticals. The affinity and binding location to HSA can significantly alter the half-life, distribution and metabolism of many drugs, thereby playing a central role in the ADME (Absorption, Distribution, Metabolism and Excretion) of many of the world's most important pharmaceuticals. However, because there have not previously been many detailed, three-dimensional studies of drug interactions and binding affinities with HSA, detailed information regarding the precise binding properties that has remained in large part unknown, and the ability to obtain and utilize this information will be extremely helpful in determining drug safety and efficacy, and in developing additional means to assess and design pharmaceutical compounds for a variety of purposes. Indeed, the major limiting factor for computer models and other processes relating to rational drug design is that they contain faulty information and may be incorrect with regard to which binding site is targeted by a particular drug compound.

Accordingly, while there are numerous patent references which relate in general to the production of computer data relating to various compounds generally unrelated to albumin and to circulatory molecules in general (see Appendix A), there are no references which relate to making detailed three-dimensional structures of the albumin binding regions so as to elicit important and useful information concerning albumin binding at those particular binding regions.

There is thus an important need to obtain additional information regarding key drug binding sites in serum albumin and to utilize that information to best determine safety and efficacy of drugs and to avoid improper and incorrect modeling by determining the correct sites for drug binding on albumin. In addition, once important binding sites are identified, it will also be possible to isolate and/or manufacture active protein fragments which maintain the binding property and activity of the site on the albumin molecule in situ so that these fragments may also be utilized in methods of evaluating and designing drugs. The ability to obtain and utilize such fragments would make commercial isolation and production of smaller fragments for use in pharmaceutical evaluation and design more commercially and technically feasible.

There is also an important need to obtain additional information regarding key drug binding sites in serum albumin and to use this information to achieve better testing with regard to drug efficacy and possible displacement reactions caused by drug activity. For example, a key to drug assessment for purposes of FDA approval is whether or not the drug significantly displaces bilirubin, a heme metabolic product that is tightly bound to albumin. However, the lack of precise knowledge of the accurate bilirubin site has led to inaccurate determinations of the likelihood that a particular drug will displace bilirubin when administered to a patient. Thus, there has been a paucity of information concerning the three-dimensional structure of albumin and an accurate picture of the binding complexes, and this has been due to the difficulty in obtaining accurate structures because of albumin's inherent conformational flexibility.

Accordingly, it will thus be important to obtain accurate three-dimensional information regarding important albumin binding sites and complexes, and this will allow utilization of such complexes in rational drug design and evaluation. In addition, an accurate identification of the binding sites of particular drugs will facilitate a determination of the likelihood of that drug displacing important biomolecules such as bilirubin, and will also allow the designing of drugs which minimize displacement of these important biomolecules. Further, such information will allow one to isolate and/or manufacture active protein fragments which maintain the binding property and activity of the site on the albumin molecule in situ so that these fragments may also be utilized in methods of evaluating and designing drugs.

There is yet another important need to further examine and elicit information concerning the binding locations to human serum albumin uniquely associated with each ligand or pharmaceutical and to create and determine the structures of protein-ligand complexes with serum albumin. In this manner, the location and study of the particular binding sites for drugs to serum albumin will be of immense predictive value to the medical and drug development community regarding drug displacement interactions. There is thus an important need in the art to obtain and utilize accurate derived three-dimensional structures of the albumin molecule in complexes with other compounds and ligands in that this information can be used for designing new pharmaceuticals with optimized albumin binding properties, e.g., increased or decreased binding, shift in albumin binding location, or other modifications to the binding affinities to achieve a beneficial result including effective drugs at lower dosages, better knowledge of drug interactions with other drugs, improved drug distribution, and reduced side effects.

### SUMMARY OF THE INVENTION

Accordingly, it is an object of the present invention to provide information regarding the three-dimensional structure and relevant binding residues at drug and ligand binding sites in the serum albumin molecule so as to provide for the first time a true picture of the molecular complexes formed between the drugs and the specific binding site and to be able to collect and utilize that information in development of effective drugs having suitable albumin binding properties.

It is a further object of the present invention to provide a method of assessing the binding of drugs at a site previously unassociated with drug binding, including the 1B region of human serum albumin, and to utilize the albumin binding information at the regions previously unknown to bind drugs in order to determine the precise nature of the binding at this site and provide a model for drug design based on albumin binding properties at those sites.

It is yet further another object of the invention to provide isolated protein fragments that contain those albumin binding sites previously unknown to have drug binding activity, including the albumin 1B binding subdomain, and to utilize said fragments in assessing drug binding activity at said sites and evaluating the safety and efficacy of drugs through their albumin binding properties at said site.

It is still another object of the present invention to provide useful three-dimensional structural information regarding albumin drug complexes at other binding sites for the purpose of improving the in vivo safety and efficacy of new drugs or existing pharmaceuticals on the basis binding properties of drugs at albumin binding sites, and further to use this information so as to be able to develop predictive capabilities in drug binding, drug displacement interactions and in silico ADME processes.

It is still further an object of the present invention to provide a method for evaluating the ability of a drug to associate with a molecule or a molecular complex comprising a human serum albumin binding region by constructing a computer model of the binding site defined by structural coordinates wherein the root mean square deviation between said structural coordinates and the structural coordinates of the albumin binding site is not more than about 1.15 Å.

These and other objects are provided by virtue of the present invention which provides for the first time an accurate method for evaluating the ability of a compound to associate with a human serum albumin binding region, such as the subdomains IA, IA/IB, IA/IIA, IB, I/II; I/III; II/III, IIA, IIA/IIB, IIB, IIIA, IIIA/IIIB, IIIB and IIIB′, by constructing a computer model of the albumin binding regions as defined by three-dimensional structural binding coordinates, such as binding residue information, wherein the root mean square deviation between the binding coordinates of said structural binding coordinates and the structural binding coordinates of the respective binding regions as set forth in Table III is not more than about 1.15 angstroms; selecting a compound to be evaluated by a method selected from the group consisting of (i) assembling molecular fragments into said compound, (ii) selecting a compound from a small molecule database, (iii) de novo ligand design of said compound, (iv) a compound obtained by modifying a compound with known binding affinity to a human serum albumin binding region; (v) a pharmaceutical or other compound as set forth in Tables I or II, below; (vi) a compound obtained by modifying a known pharmaceutical compound, or active portion thereof, of human serum albumin; employing computational means to perform a fitting program operation between computer models of the said compound to be evaluated and said binding region in order to provide an energy-minimized configuration of the said compound in the albumin binding region; and evaluating the results of said fitting operation to quantify the association between the said compound and the binding region computer model, thereby evaluating the ability of the said compound to associate with the albumin.

In addition, in accordance with the present invention, it has now been learned that certain binding regions of human serum albumin which heretofore have not been known to bind bioactive drugs, such as subdomain 1B, do in fact act as a drug binding site. In fact, the present inventors have now discovered that subdomain 1B is in fact the major site for the binding of therapeutic drug compounds which is a surprising result considering that this site was not previously known to be a drug binding site at all. Further, other sites appeared to have some binding affinity for non-drugs such as gases such as propofol (site IIIA, IIIB), or halothane (e.g., IIA-IIB, etc.), but in none of these cases were any of these sites thought to be a binding location for drugs. Accordingly, in accordance with the present invention, these sites with newly discovered drug activity can be utilized in methods of assessing safety and efficacy of drugs binding at those sites, and can determine the likelihood that a particular drug will displace other drugs or important biomolecules at a particular binding site not previously thought to bind to therapeutic drugs.

In this regard, it is thus possible to prepare protein fragments which contain the particular subdomain binding region and to utilize these fragments in methods of assessing albumin binding properties of particular drugs. In addition, it is also possible to prepare modified albumins having one or more particular contemplated that the reference to human serum albumin as set forth herein also includes any such analogs, derivatives, etc., or other serum albumin from other species which has the same or similar binding characteristics with regard to the specific binding regions disclosed herein.

In accordance with the present invention, the characteristic binding locations of human serum albumin were determined using detailed X-ray crystallography at a very high resolution to obtain a three-dimensional view of the albumin molecule and the atomic complexes formed by the interaction of albumin with a series of important pharmaceutical compounds. These investigations focused on more than 100 clinically approved pharmaceuticals based on high plasma binding and/or high affinity to HSA. This initial screening of clinical pharmaceuticals resulted in an initial list of 350 targeted pharmaceuticals and a few selected drug-like molecules of interest. As indicated above, there previously had been a paucity of three-dimensional drug binding data in the literature which reflected prior difficulties in obtaining such data due to albumin's inherent conformational flexibility. A complete description of the structural determination of a serum albumin protein through crystallographic means is set forth in Nature, Vol. 358:209 (July 1992), incorporated herein by reference. However, the previous determinations of the serum albumin structure gave little insight into its binding locations, and a number of binding regions in human serum albumin were not considered to involve drug binding and thus have been ignored in terms of interest and computer modeling dealing with drug interactions. For example, prior references dealing with in silico prediction of drug-binding involving human serum albumin did not recognize that drugs bound at site IB, and thus had flawed modeling based on this erroneous assumption. See, e.g., Colmenarejo, Medicinal Research Reviews, Vol. 23 (3) 275-301 (2002), incorporated herein by reference. To the contrary, as indicated below, the present inventors have now discovered that numerous albumin binding regions, including subdomains IA, IA/IB, IA/IIA, IB, I/II; I/III; II/III, IIA/IIB, IIB, IIIA/IIIB, IIIB and IIIB′, all act as binding sites for drugs, and that site 1B fragments actually appears to be the major site for drug binding on human serum albumin. Thus, as indicated below, these sites can all be utilized in assessing drug interactions at those sites in a manner not before possible.

As indicated above, this invention involves the use of the atomic coordinates of serum albumin for the application of improving the in-vivo efficacy or safety of newly developing or existing pharmaceuticals.

In particular, the invention relates to a method for evaluating the ability of a compound to associate with a molecule or molecular complex comprising a human serum albumin binding region selected from the group consisting of binding subdomains IA, IA/IB, IA/IIA, IB, I/II; I/III; II/III, IIA, IIA/IIB, IIB, IIIA, IIIA/IIIB, IIIB and IIIB′, said method comprising the steps of:

a) constructing a computer model of said binding region defined by three-dimensional structural binding coordinates wherein the root mean square deviation between said structural binding coordinates and the structure binding coordinates of the respective binding region as set forth in Table S1 is not more than about 1.15 angstroms;

b) selecting a compound to be evaluated by a method selected from the group consisting of (i) assembling molecular fragments into said compound, (ii) selecting a compound from a small molecule database, (iii) de novo ligand design of said compound, (iv) a compound obtained by modifying a compound with known binding affinity to a human serum albumin binding region; (v) a pharmaceutical or other compound as set forth in Tables I or II; (vi) a compound obtained by modifying a known pharmaceutical compound, or active portion thereof, of human serum albumin

c) employing computational means to perform a fitting program operation between computer models of the said compound to be evaluated and said binding region in order to provide an energy-minimized configuration of the said compound in the binding region; and

d) evaluating the results of said fitting operation to quantify the association between the said compound and the binding region computer model, thereby evaluating the ability of the said compound to associate with the said binding region.

Alternatively, the root mean square deviation can be slightly larger, e.g., within about 2.5 angstroms, 2.7 angstroms or 3.0 angstroms, and still provide meaningful information to assess drug interactions as set forth below. The psi angle may be in the range of about −30 to +30 degrees, or in the range of about −60 to +120 degrees.

Other methods and applications of the invention are described further below as well.

In particular, the invention relates to obtaining information about the three-dimensional structures of drugs that bind to human serum albumin at one or more binding sites on albumin, including binding regions IA, IA/IB, IA/IIA, IB, I/II; I/III; II/III, IIA, IIA/IIB, IIB, IIIA, IIIA/IIIB, IIIB and IIIB′. While these regions themselves are known, no one has previously conducted detailed three-dimensional structural analysis of these sites so as to provide a picture of the structural coordinates which reveal particular positions of the albumin molecule wherein binding takes place. As a result, with the information learned with regard to the particular structure of the binding regions as set forth below with regard to these regions, a truer picture of the nature of drug-albumin binding has emerged, and this information will be useful for the assessment and designing of drugs.

In one aspect of the invention, the drug complexes and the structural information necessary to assess drug interactions in accordance with the inventions fall generally into the following sites having the following structural contacting residues:

Site IB:

F036, F037, P110, N111, L112, P113, R114, L115, V116, R117, P118, V122, M123, A126, T133, F134, L 135, K137, Y138, Y140, E141, E142, R145, H146, F149, L154, F157, A158, Y161, F165, L182, D183, L185, R186, G189, K190, S193

Site IIA:

F149, Y150, E153, S192, K195, Q196, L198, K199, C200, S202, F211, W214, A215, R218, L219, R222, F223, L238, V41, H242, R257, L260, A261, I264, K286, S287, H288, I290, A291, V343, D451, Y452, V455

Site IIIA:

R383, P384, L387, I388, N391, C392, F395, F403, L407, R410, Y411, K414, V415, V418, L423, V426, S427, L430, G431, V433, G434, S435, C437, C438, M446, A449, E450, L453, V456, L457, L460, V473, R484, R485, F488, S489, L491

Site IIA-IIB:

R209, A210, A213, W214, D324, L327, G328, L331, L347, A350, K351, E354, S480, L481, V482

Site IA:

V007, F019, V023, F027, E045, V046, F049, A050, E060, N061, K064, L066, L069, F070, G071, D072, K073, C075, T076, C091, R098, L251

In accordance with the invention, methods for evaluating the ability of a compound to associate with a molecule or molecular complex comprising a human serum albumin binding region selected from the group consisting of binding subdomains IA, IA/IB, IA/IIA, IB, I/II; I/III; II/III, IIA, IIA/IIB, IIB, IIIA, IIIA/IIIB, IIIB and IIIB′, will utilize the information above with regard to the structural binding coordinates at the contacting residues set forth above, and as set forth in the Table S1 below. It is contemplated in accordance with the invention that there may be distinct subsets of coordinates based on a subset of contacting positions as set forth herein, and thus in the practice of the invention, the constructing a computer model of one or more binding regions defined by three-dimensional structural binding coordinates as set forth herein, where the root mean square deviation between said structural binding coordinates and the structure binding coordinates of the respective binding region as set forth in Table S1 is not more than about 1.15 angstroms, refers to coordinates at both the particular contacting residues set forth herein, or sufficient numbers of residues which would provide the same information.

In addition to the information concerning the general binding regions as set forth herein, a number of drug complexes have been subject to the present method to provide information regarding structural coordinates and binding residues so as to be useful in conjunction with the invention. The methods and materials used to create such computer databases are well known in the art and are discussed further below. In particular, the following Tables 1 and 2 disclose the complexes for which structural contact residue information has been obtained in accordance with the invention.

TABLE I
PHASE I - Drug Binding Locations for Specific Drugs

| Compound or Drug | Albumin Binding Location |
|---|---|
| 59. NCP007 | IIA |
| 60 NCP008 | IIA |
| 61 NCP012 | IB, IIA |
| 62 NCP023 | IB |
| 63. NCP024 | IIA |
| 64. NCP049 | IB, IIA |
| 65. NCP 051 | IB, IIA |
| 66. Bupropion | IIA, IIIA |
| 67. Beta-Estradiol | IB, IIA |
| 68. Prednisone | IIA |
| 69. Penicillin V | IIA |
| 70. Phenytoin | IIA |
| 71. Lovastatin | IIA |
| 72. Mupirocin | IA |
| 73. Celocoxib | IIA |
| 74. Losartan | IB |
| 75. Amlodipine Besylate | IIA, IIIA |
| 76. Chlorpropamide | IIA, IIIA |
| 77. A77 1726 | IB |
| 78. Terbinafine | IIIA |
| 79. Ceftriaxone | IB |
| 80. Pioglitazone | IIA, IIIA |
| 81. Estradiol Valerate | IIA |
| 82. R-Etodolac | IB |
| 83. Haloperidol | IA |
| 84. Indomethacin | IB |
| 85. Tertinoin | IIIA |
| 86. Methayclothiazide | IIA-IIB |
| 87. Donepezil | IIA |
| 88. Bupivacaine | IB |
| 89. Nefazodone | IIIA |

TABLE II
PHASE II: Contacting Residues involved in Drug-Albumin
Complexes: (for each drug and its indication)

| | |
|---|---|
| 1. | for the pharmaceutical Meloxicam, a COX-2 inhibitor: |
| | Site IIA, Y150, E153, K199, F211, W214, A215, R218, L219, R222, |
| | L238, V241, H242, R257, L260, I264, S287, I290, A291 |
| 2. | for the pharmaceutical Ethinyl-Estradiol, for hormone replacement: |
| | Site IB, L115, V116, R117, P118, M123, A126, F134, K137, Y138, |
| | E141, Y161, F165, L182, R186 |
| 3. | For 6-MNA {Nabumetone}, an NSAID: |
| | Site IB, R114, L115, R117, M123, L135, Y138, L139, I142, H146, |
| | F149, L154, F157, A158, Y161, F165, L182, L185, R186, G189, |
| | K190, |
| | Site IIA-IIBR209, A210, A213, W214, D324, L327, G328, L331, |
| | L347, A350, K351, L481, V482 |
| | Site IIIA, L387, I388, N391, C392, F403, L407, R410, Y411, K414, |
| | L430, V433, G434, C437, A449, L453, R485, F488, S489 |
| 4. | For Naproxen, an NSAID: |
| | Site IB, L115, V116, R117, P118, M123, L135, Y138, L139, I142, |
| | H146, F149, L154, F157, A158, Y161, F165, L182, L185, R186, |
| | G189, K190 |
| | Site IIA-IIB, R209, A210, A213, D324, L327, G328, L331, L347, |
| | A350, K351, E354, S480, L481, V482 |
| | Site IIIA, L387, I388, N391, C392, F403, L407, R410, Y411, K414, |
| | L430, V433, G434, C437, C438, A449, L453, L457, R485, F488, |
| | S489 |
| 5. | For Diclofenac, an NSAID: |
| | Site IIIA, P384, L387, I388, N391, V392, F403, L407, R410, Y411, |
| | L430, V433, G434, C438, A449, E450, L453, R485, S489 |
| 6. | For Etodolac, an NSAID: |
| | Site IB, L115, Y138, E141, I142, R145, H146, Y161, L182, L185, |
| | R186, G189, K190 |
| | Site IIIA, P384, L387, I388, N391, F403, L407, R410, Y411, K414, |
| | L430, V433, A449, E450, L453, L457, R485, S489 |
| 7. | For S-Etodolac, an NSAID: |
| | Site IIIA, P384, L387, I388, N391, F403, L407, R410, Y411, K414, |
| | L430, V433, A449, L453, L457, R485, S489 |
| 8. | For Ketorolac, an NSAID: |
| | Site IB, L115, R117, Y138, I142, H146, F157, Y161, L182, D183, |
| | L185, R186, G189, K190, S193 |
| | Site IIIA, L387, I388, N391, C392, F403, L407, R410, Y411, K414, |
| | L430, V433, G434, C438, A449, L453, R485, F488, S489 |
| 9. | For R-Ketorolac, an NSAID: |
| | Site IB, R117, Y138, I142, H146, F149, L154, F157, Y161, L182, |
| | D183, L185, R186, G189, K190, S193 |
| 10. | For S-Ketorolac, an NSAID: |
| | Site IIIB, F507, F509, I513, L516, R521, K524, K525, A528, L532, |
| | V547, M548, F551, A552, V555 |
| 11. | For Diflunisal, an analgesic: |
| | Site IIIA, L387, I388, N391, F403, L407, R410, Y411, K414, L430, |
| | V433, G434, A449, C438, L453, L457, R485, F488, S489 |
| 12. | For Mefenamic Acid, an analgesic: |
| | Site IIIA, E383, P384, L387, N391, C392, F403, L407, R410, Y411, |
| | L430, V433, G434, C438, A449, E450, L453, R485, S489 |
| 13. | For Ibuprofen, an analgesic: |
| | Site IIIA, L387, R410, Y411, K414, V415, V418, L423, V426, |
| | L430, L453, L457, L460, R485, F488, S489, L491 |
| | Site IIIB, Y401, K402, N405, F507, F509, K525, Q526, A528, L529, |
| | L532, K545, V547, M548, D549, F551, A552 |
| 14. | For S-Ibuprofen, an analgesic: |
| | Site IB, L115, V116, R117, P118, M123, F134, Y138, I142, |
| | Y161, F165, L182, D183, R186 |
| | Site IIIA, L387, N391, R410, Y411, K414, V415, L423, V426, |
| | L430, L453, L457, L460, F488, S489 |
| 15. | For the pharmaceutical Flurbiprofen, an analgesic |
| | Site IIIA, L387, I388, N391, C392, F395, F403, L407, R410, Y411, |
| | K414, L430, V433, G434, C438, A449, L453, L457, L485, F488, |
| | S489 |
| 16. | For the pharmaceutical Salsalate, an analgesic |
| | Site IIIB, F509, I513, R521, K524, K525, A528, M548, F551, A552, |
| | V555 |
| 17. | For the pharmaceutical Piroxicam, an analgesic |
| | Site IIA, K195, Q196, L198, K199, F211, W214, A215, R218, L219, |
| | R222, L238, H242, A291, V343, D451, Y452, V455 |
| 18. | For the pharmaceutical Propoxyphene, an analgesic |
| | Site IIIB, F502, F507, F509, A528, E531, L532, H535, V547, D550, |
| | F551, F554, G572, L575, V576, A578, S579 |
| 19. | For the pharmaceutical Aspirin, an analgesic |
| | Site IIIB, F509, I513, R521, K524, K525, A528, M548, F551, A552, |
| | V555 |
| 20. | For the pharmaceutical Buspirone, an anti-anxiety |
| | Site IIIA, L387, I388, N391, C392, F403, L407, R410, Y411, K414, |
| | V415, V418, L423, V426, L430, G434, S435, C438, A449, L453, |
| | L457, L460, R485, F488, S489 |
| 21. | For the pharmaceutical Zafirlukast, an anti-bacterial |
| | Site IIA, Y150, E153, K195, L198, K199, S202, F211, W214, A215, |
| | R218, L219, R222, L238, V241, H242, R257, L260, A261, I264, |
| | S287, H288, I290, A291, D451, Y452, V455 |
| 22. | For the pharmaceutical Sulfisoxazole, an anti-bacterial |
| | Site IB, L115, V116, R117, P118, M123, Y138, I142, Y161, L182, |
| | L185, R186, G189 |
| 23. | For the pharmaceutical Sulbenicillin, an anti-bacterial |
| | Site IIA, Y150, K195, K199, W214, R218, L219, R222, L238, H242, |
| | R257, L260, A261, I264, S287, I290, A291 |
| 24. | For the pharmaceutical Penecillin G, an anti-bacterial |
| | Site IIA, Y150, K195, K199, F211, W214, A215, R218, L219, R222, |
| | L238, H242, R257, L260, I264, S287, I290, A291 |
| 25. | For the pharmaceutical Camptothecin, anti-cancer |
| | Site IB, L115, V116, R117, M123, Y138, I142, H146, F149, L154, |
| | F157, Y161, L182, L185, R186, G189, K190, S193 |
| 26. | For the pharmaceutical 9 Nitro-Camptothecin, anti-cancer |
| | Site IB, L115, V116, R117, Y138, I142, H146, F149, L154, F157, |
| | Y161, L182, D183, L185, R186, G189, K190, S193 |
| 27. | For the pharmaceutical Idarubicin, anti-cancer |
| | Site IB, F036, F037, L115, V116, R117, P118, V122, M123, A126, |
| | T133, F134, L135, K137, Y138, Y140, E141, I142, Y161, F165, |
| | L182 |
| 28. | For the pharmaceutical Bicalutamide, anti-cancer |
| | Site IB, L115, V116, R117, P118, V122, M123, A126, F134, K137, |
| | Y138, E141, Y161, F165, L182, R186 |
| 29. | For the pharmaceutical Teniposide, anti-cancer |
| | Site IB, L115, V116, R117, P118, A126, F134, K137, Y138, E141, |
| | I142, R145, H146, F149, F157, Y161, L182, D183, L185, R186, |
| | G189, K190 |
| 30. | For the pharmaceutical NCP014, anti-coagulant (cand.) |
| | Site IB, P110, N111, L112, P113, R114, L115, I142, R145, H146, |
| | F149, F157, Y161, L185, R186, G189, K190 |
| | Site IIIA, F149, Y150, E153, S192, K195, Q196, K199, C200, L238, |
| | V241, H242, C245, C246, C253, R257, L260, A261, I264, S287, |
| | I290, A291 |
| 31. | For the pharmaceutical NCP023 |
| | Site IB, L115, E141, I142, R145, H146, F149, L154, F157, Y161, |
| | L185, R186, G189, K190 |
| 32. | For the pharmaceutical R-Warfarin |
| | Site IIA, F211, W214, A215, R218, L219, R222, F223, L238, V241, |
| | H242, R257, L260, A261, I264, S287, I290, A291 |
| 33. | for the pharmaceutical Chloro-warfarin |
| | Site IIA, Y150, K195, Q196, K199, F211, W214, A215, R218, L219, |
| | R222, F223, L238, H242, L260, I265, I290, A291 |
| 34. | For the pharmaceutical Citalopram |
| | Site IIIB, F502, F507, L532, V533, H535, K536, A539, T540, Q543, |
| | L544, V547, F551, L575, V576, A577 |
| 35. | For the pharmaceutical Glyburide |
| | Site IB, L115, V116, R117, P118, M123, A126, F134, K137, Y138, |
| | E141, I142, H146, F149, Y161, L182, D183, L185, R186, G189, |
| | K190 |
| 36. | For the pharmaceutical Glipizide |
| | Site IB, L115, V116, R117, P118, M123, A126, F134, K137, Y138, |
| | E141, I142, H146, F149, Y161, L182, D183, L185, R186, G189, |
| | K190 |
| 37. | For the pharmaceutical Promethazine |
| | Site IIB, D308, F309, N318, E321, A322, V325, F326, N329 |
| 38. | For the pharmaceutical Terazosin |
| | Site IB, L115, V116, R117, P118, M123, A126, F134, K137, Y138, |
| | E141, I142, H146, F149, L154, F157, Y161, L182, L185, R186, |
| | G189, K190, S193 |
| 39. | For the pharmaceutical Ramipril |
| | Site IB, L115, V116, R117, P118, M123, Y138, L139, E141, I142, |
| | H146, F149, L154, F157, A158, Y161, L182, L185, R186, G189, |
| | K190, S193 |
| 40. | For the pharmaceutical Prazosin |
| | Site IB, L115, V116, R117, P118, M123, F134, K137, Y138, I142, |
| | H146, F149, Y161, L182, L185, R186, G189, K190, S193 |
| 41. | For the pharmaceutical Telmisartan |
| | Site IB, L115, V116, R117, P118, M123, F134, K137, Y138, E141, |
| | I142, R145, H146, F149, Y161, F165, L182, L185, R186, G189, |
| | K190, |
| 42. | For the pharmaceutical Hydralazine |
| | Site IB, Y138, I142, H146, F149, F157, Y161, L182, L185, R186, |
| | G189, K190 |
| 43. | For the pharmaceutical Carvedilol |
| | Site IIIA, P384, L387, I388, N391, F403, L407, R410, Y411, K414, |
| | V415, L430, V433, A449, E450, L453, L457, L460, R485, F488, |
| | S489, L491 |
| 44. | For the pharmaceutical?-Carvedilol |
| | Site IIIA, P384, L387, I388, N391, F403, L407, R410, Y411, K414, |
| | V415, V426, L430, V433, A449, E450, L453, L457, L460, R485, |
| | F488, S489 |
| 45. | For the pharmaceutical Candesartan Cilexitil |
| | Site II-III, K195, L198, K199, S202, L203, F206, G207, A210, F211, |
| | W214, V344, L347, E450, L453, S454, L457, E479, S480, L481, |
| | R484, R485 |
| | Site IIIA, P384, L387, I388, N391, C392, F395, F403, L407, Y411, |
| | K414, V415, L423, V426, L430, V433, G434, C438, M446, A449, |
| | L453, V456, L457, L460, V473, R484, R485, F488, S489, L491 |
| | Site IIIA-IIIB, L398, Y401, K402, N405, A406, V409, R410, L529, |
| | A539, T540, K541, E542, L544, K545, M548 |
| 46. | For the pharmaceutical Oxaprozin |
| | Site IIIA, P384, L387, I388, N391, C392, F403, L407, R410, Y411, |
| | K414, L430, V433, G434, C437, C438, A449, E450, L453, R485, |
| | S489 |
| 47. | For the pharmaceutical Sulfasalazine |
| | Site IIA, Y150, K195, L198, K199, S202, F211, W214, A215, R218, |
| | L219, R222, L238, H242, R257, L260, A261, I264, K286, S287, |
| | H288, I290, A291, V343 |
| 48. | For the pharmaceutical Risperidone |
| | Site IIIA, L387, I388, N391, C392, F403, L407, R410, Y411, K414, |
| | V415, L430, G431, V433, G434, C437, C438, A449, L453, L457, |
| | L460, R485, F488, S489 |
| 49. | For the pharmaceutical Meclofenamic Acid, anti-pyretic |
| | Site IIIA, P384, L387, I388, N391, C392, F403, L407, R410, Y411, |
| | L430, V433, G434, C437, C438, A449, E450, L453, R485, S489 |
| 50. | For the pharmaceutical Balsalazide, anti-ulcerative |
| | Site IIA-IIIB, S401, K402, N405, A406, L407, V409, R410, K413, |
| | K525, K541, L544, K545, M548, D549, A552 |
| 51. | For the pharmaceutical Pantoprazole |
| | Site IIA, Y150, K195, L198, K199, S202, F211, W214, A215, R218, |
| | L219, R222, F223, L238, H242, R257, 260, A261, I264, S287, |
| | I290, A291 |
| 52. | For the pharmaceutical Oxazepam, anxiolytic |
| | Site IIIA, P384, L387, I388, N391, C392, F403, L407, R410, Y411, |
| | L430, V433, G434, C438, A449, E450, L453, R485, S489 |
| 53. | For the pharmaceutical Gemfrirozil, Ch'rol Lowering |
| | Site IB, L115, V116, R117, F134, L135, Y138, L139, I142, R145, |
| | H146, F149, L154, F157, A158, Y161, F165, L182, L185, R186, |
| | G189, K190, S193 |
| | Site IIIA, L387, R410, Y411, K414, V415, V418, L423, V426, S427, |
| | L430, L453, L457, L460, V473, R485, F488, S489, L491 |
| 54. | For the pharmaceutical Fluvastatin, Ch'rol Lowering |
| | Site IIA, Y150, K195, L198, K199, S202, F211, W214, A215, R218, |
| | L219, R222, L238, H242, R257, A291, D451, Y452, V455 |
| | Site IIIA, P384, L387, I388, N391, C392, F403, L407, R410, Y411, |
| | K414, L430, V433, G434, G438, M446, A449, E450, L453, R485, |
| | F488, S489 |
| 55. | For the pharmaceutical Clofibric, cholesterol (ch'rol) lowering |
| | Site IB, I142, R145, H146, F149, F157, L185, R186, G189, K190 |
| | Site IIIA, L387, I388, N391, C392, F403, L407, R410, Y411, K414, |
| | L430, V433, G434, C438, A449, L453, R485, F488, S489 |
| 56. | For the pharmaceutical Chlorothiazide: |
| | Site IB, R114, L115, R117, Y138, E141, I142, R145, H146, F149, |
| | Y161, L182, L185, R186, G189, K190 |
| 57. | For the ligand Bilirubin |
| | Site IB, R114, L115, V116, R117, P118, V122, M123, F134, Y138, |
| | I142, H146, F149, L154, F157, Y161, F165, L182, L185, R186, |
| | G189, K190 |
| 58. | For the pharmaceutical Hemin |
| | Site IB, R114, L115, V116, R117, P118, M123, F134, L135, Y138, |
| | L139, I142, H146, F149, L154, F157, A158, Y161, R186, G189, |
| | K190, S193 |

In accordance with the present invention, it is thus an object of the invention to provide a method of producing a computer readable database comprising the three-dimensional molecular structural coordinates of one or more human albumin binding regions selected from the group consisting of the binding region IA, IA/IB, IA/IIA, IB, I/II; I/III; II/III, IIA, IIA/IIB, IIB, IIIA, IIIA/IIIB, IIIB and IIIB′, said method comprising a) obtaining three-dimensional structural coordinates defining said binding regions; and b) introducing said structural coordinates into a computer to produce a database containing the molecular structural coordinates of said binding regions, and a computer database as produced by this method. These databases can be obtained and produced using technology readily available to one skilled in the art, and specific programs useful in the invention are set forth below. It is also contemplated that one skilled in the are will be able to utilize this structural database for a variety of assessments and predictions with regard to drug interaction and development, including using the structural information stored in the database for in silico methods of predictive ADME. The albumin sequence information with regard to the above binding regions is well known, and the specific sequences have the following residues for the various domains: Domain I (1 through 192); Domain II (193 through 395);Domain III (396 THROUGH 585); Subdomain: IA (1-105), IB (106-192), IIA (193-291), IIB (292-395), IIIA(396491), IIIB (492-585).

Similarly, another method is provided in accordance with the invention which involves producing a computer readable database comprising a representation of a compound capable of binding one or more human albumin binding subdomains, said method comprising a) introducing into a computer program a computer readable database produced by the method above; b) generating a three-dimensional representation of one or more human albumin binding subdomains in said computer program; c) superimposing a three-dimensional model of at least one binding test compound on said representation of said one or more binding subdomains; d) assessing whether said test compound model fits spatially into one or more human serum albumin binding subdomains; and e) storing a structural representation of a compound that fits into one or more human serum albumin binding subdomains. Once again, the present invention is related to computer databases generated by such methods, and further involves utilizing the structural representations stored in said database for predictive ADME and other uses based on drug interactions with albumin.

Further, the present invention can be used in methods of assessing drugs when dealing with circulatory interfaces. In particular, the nature of ligand binding to serum albumin, eg., site location, affinity, etc., is thought to play a role in the distribution of certain drugs and endogenous ligands across organ circulatory interfaces such as the liver, kidney and brain. An improved understanding of these important, but poorly understood properties of albumin, as enabled by the current invention, can be then be used to tune the pharmacokinetic properties of both newly developing and existing pharmaceuticals leading to safer and more efficacious drugs.

In another aspect of the present invention, the present inventors have discovered numerous albumin binding regions wherein drug interactions take place, and these regions can be utilized in a number of ways to assess the effects of the particular nature of the drug binding on the safety and efficacy of the drug. For example, it was long thought that drugs did not bind to site IB of serum albumin which is a site for bilirubin and numerous other biomolecules and endogenous ligands. Accordingly, when assessing the likelihood of a given drug causing displacement of bilirubin, it was not thought to check if the drug bound at the albumin IB site or at another site. However, with the knowledge that IB is an important binding site for many drugs, this information can now be utilized as a further test or screening to see if a drug will cause displacement of a biological molecule at a particular site (such as bilirubin at binding site IB). Accordingly, the present invention relates to the utilization of these newly discovered drug binding sites in methods of assessing the likelihood for drugs to displace biomolecules or other compounds at a given albumin binding site.

The present invention is thus concerned with methods of utilizing information obtained by virtue of the structural information learned from a detailed three-dimensional analysis of the albumin binding regions which has provided information concerning the contacting residues with regard to those binding regions. In one such method, a method for evaluating the ability of a compound to associate with a molecule or molecular complex comprising a human serum albumin binding region selected from the group consisting of binding subdomains IA, IA/IB, IA/II, IB, I/II; I/III; II/III, IIA, IIA/IIB, IIB, IIIA, IIIA/IIIB, IIIB and IIIB′ is provided which comprises:

a) constructing a computer model of said binding region defined by three-dimensional structural binding coordinates wherein the root mean square deviation between said structural binding coordinates and the structure binding coordinates of the respective binding region as set forth in Table S1 is not more than about 1.15 angstroms; b) selecting a compound to be evaluated by a method selected from the group consisting of (i) assembling molecular fragments into said compound, (ii) selecting a compound from a small molecule database, (iii) de novo ligand design of said compound, (iv) a compound obtained by modifying a compound with known binding affinity to a human serum albumin binding region; (v) a pharmaceutical or other compound as set forth in Tables I or II; (vi) a compound obtained by modifyihg a known pharmaceutical compound, or active portion thereof, of human serum albumin; c) employing computational means to perform a fitting program operation between computer models of the said compound to be evaluated and said binding region in order to provide an energy-minimized configuration of the said compound in the binding region; and d) evaluating the results of said fitting operation to quantify the association between the said compound and the binding region computer model, thereby evaluating the ability of the said compound to associate with the said binding region. The level of the root mean square deviation in these evaluation methods can vary and still provide a useful product, and thus it is possible for the deviation to be on the order of 2.5, 2.7, or 3.0 angstroms, for example. In this method, the psi angle can range from about −30 degrees. to +30 degrees, and the phi angle can be in the range of about 60 degrees to 120 degrees.

Again, as indicated above, such method steps involve computational skills using techniques and technology well within the skill of one of ordinary skill in this art as set froth herein. Still other methods contemplated by the present invention involve identifying an activator or inhibitor of a molecule comprising a human serum albumin binding region selected from the group consisting of binding region IA, IA/IB, IA/IIA, IB, I/II; I/III; II/III, IIA, IIA/IIB, IIB, IIIA, IIIA/IIIB, IIIB and IIIB′ using the steps of constructing a computer model of the binding region defined by three-dimensional structural binding coordinates as set forth above, selecting a compound to be evaluated as set forth above, employing computational means to perform a fitting program operation between computer models of the said compound to be evaluated and said binding region in order to provide an energy-minimized configuration of the said compound in the binding region; evaluating the results of said fitting operation to quantify the association between the said compound and the binding region computer model, thereby evaluating the ability of the said compound to associate with the said binding region; then synthesizing said compound; and contacting said compound with said molecule to determine the ability of said compound to activate or inhibit said molecule. The synthesis of the compound resulting from these steps can thus be conducted in conventional ways using technology available to one skilled in the art.

Another method in accordance with the invention is to identify ligand interaction at the human serum albumin binding regions as described above, using the constructing, selecting, computational and evaluating steps as set forth above to evaluating the ability of a test compound to associate with a given binding region. This can be followed up by synthesizing said compound; and contacting said compound with said molecule so as to determine the ability of said ligand interact with said molecule if needed. Still further, it is possible to utilize the above steps to optimizing the binding of a compound to a human serum albumin binding region and evaluate the results of said fitting operation to optimize the binding characteristics of said compound to an albumin binding site. By optimization is meant those techniques used to maximize the safety and efficacy of drug interactions that involve albumin binding, whether increasing the binding affinity when necessary, or designing a drug to have a lesser binding affinity to a particular site when necessary to avoid potential harmful side effects such as displacement of a useful biomolecule, e.g., bilirubin.

Accordingly, displacement studies in accordance with the invention can be carried out, for example, using one compound with known binding sites and affinity as a molecular probe to test other compounds' binding site and affinity. E.g., if the known compound can be displaced from its binding site by the testing compound, then the testing compound is binding to the same site, and the relative binding affinity can also be obtained. Suitable methods in accordance with the present invention would include ultrafiltration (e.g., from Millipore), albumin columns, and any other suitable techniques used by those skilled in the art. The invention can also be used as a comparison model. For example, using albumin-binding column, the retention time can be used as a comparison to calculate binding the binding affinity of the testing compound.

Accordingly, in addition to the above drug displacement methods, it is contemplated that the present invention will be useful in the obtaining the computer database or “databank” information as set forth above, or the individual specific binding information as provided herein, eg., in Tables I, II and III, and using this information in drug displacement methods as well. This would include displacement for 1) improving the therapeutic concentration of drug for efficacy and safety reasons; 2) predicting undesirable drug interactions (such as the bilirubin but also other drug displacement—such as now important with the growing baby boomer population where people are on several medications and 3) or to effect a lower dosage of drugs in various drug combinations.

In light of the discovery that certain binding regions are sites for drug interactions, the present invention also contemplates the isolation and use of protein fragments containing these binding subdomains from human serum albumin, namely those binding subdomains including binding regions IA, IA/IB, IA/IIA, IB, I/II; I/III; II/III, IIA/IIB, IIB, IIIA/IIIB, IIIB and IIIB′.

Moreover, these fragments cane be utilized to determining the binding affinity of a drug to a target human serum albumin binding subdomain selected from the group consisting of human binding subdomain selected from the group consisting of binding region IA, IA/IB, IA/IIA, IB, I/II; I/III; II/III, IIA/IIB, IIB, IIIA/IIIB, IIIB and IIIB′ by isolating a protein fragment containing one of these regions, introducing that protein fragment to a drug in an amount and for a time sufficient to block the site on that drug that will bind to the target albumin binding subdomain, and then determining the level of human serum albumin binding of the drug following said introduction of said protein fragment in order to determine the binding affinity of the drug to the target albumin binding subdomain. Moreover, this method can be further used to assess the likelihood that the drug will displace a molecule or compound at the target binding subdomain, with the knowledge of the drug's binding site making it more likely it will displace a drug at that binding site.

It is also possible to provide kits for performing such tests, and in general, these kits will include conventional materials for conducting and monitoring reactions, and normally will include the protein fragment containing the binding subdomain selected from the group consisting of binding region IA, IA/IB, IA/IIA, IB, I/II; I/III; II/III, IIA/IIB, IIB, IIIA/IIIB, IIIB and IIIB′ in an amount sufficient to block the site on a drug that would bind to a human serum albumin binding domain, a means to allow the introduction of the isolated fragment to a drug being assessed, and means to assess the binding of human serum albumin to the drug following introduction of the isolated fragment for a time sufficient to allow binding to take place. These items will conventional include means to determine that binding has taken place, such as radioactive isotopes, enzymes, colorimetric indicators, etc., as would be readily understood by one skilled in this art.

It is also possible to utilize a modified human serum albumin that has a particular binding site blocked so as to test a drug for its ability to bind to that site. This method would be carried out by obtaining a human serum albumin having a target binding subdomain that is blocked, introducing the “blocked” albumin to the drug of interest, and then determining the level of binding of the drug to the human serum albumin with a blocked target binding subdomain, and using this information to assess the binding affinity of the drug to the target albumin binding subdomain. Once again, this information could be used to assess the likelihood that the drug will displace a molecule or compound at the target binding subdomain, and this may be carried out using a kit including a human serum albumin having a target binding subdomain that is blocked, a means to allow the introduction of the blocked human serum albumin to a drug being assessed, and means to assess the binding of the blocked human serum albumin to the drug being assessed.

When preparing fragments containing the specific binding regions of the present invention, it will be well understood by those skilled in the art that a number of alternate sequences can be prepared which will differ in some slight manner from the binding regions as discussed above, yet which are considered within the scope of the invention. For example, these alternate embodiments include those fragments or sequences which have slight variations as to specific amino acids, such as those which include an addition or deletion of a particular amino acid, possibly at the leading or trailing end of the fragment, which maintain the binding properties of the albumin family of proteins in the manner set forth above. Additionally, those sequences which contain certain changes in specific amino acids which may enhance or decrease the binding affinity of various compounds, or reduce the likelihood of producing an antigenic response, will also be within the scope of the invention as would be obvious to one of ordinary skill in the art. Finally, as set forth above, it is contemplated that because the subdomain regions of the multigene family of albumin proteins appear to be the same or similar, the biologically active protein fragments of the present invention can be constructed from specific binding regions of any of the proteins of the serum albumin family, such as the Gc and AFP proteins discussed above. All of these embodiments are deemed to be covered within the scope of the present invention which is set forth in the claims appended hereto.

In general, as with regard to the above methods, the fragments of the invention can be used for a variety of applications including crystallographic and NMR drug and ligand binding studies (structural studies), microcalorimetery (drug-binding affinity and locations determination), mass spectroscopy, and therapeutic (drug delivery) as well as many other applications.

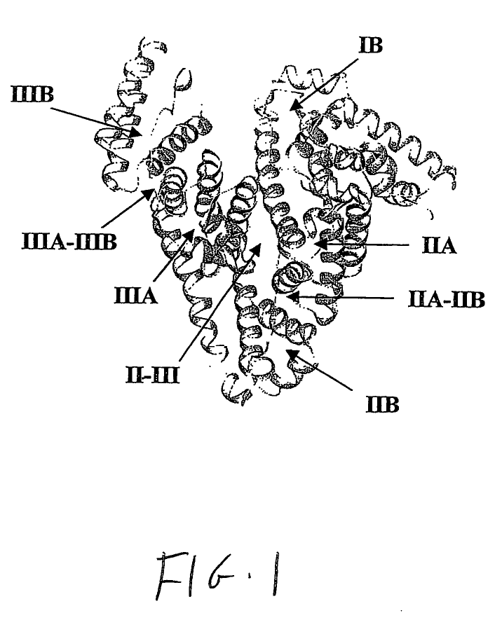

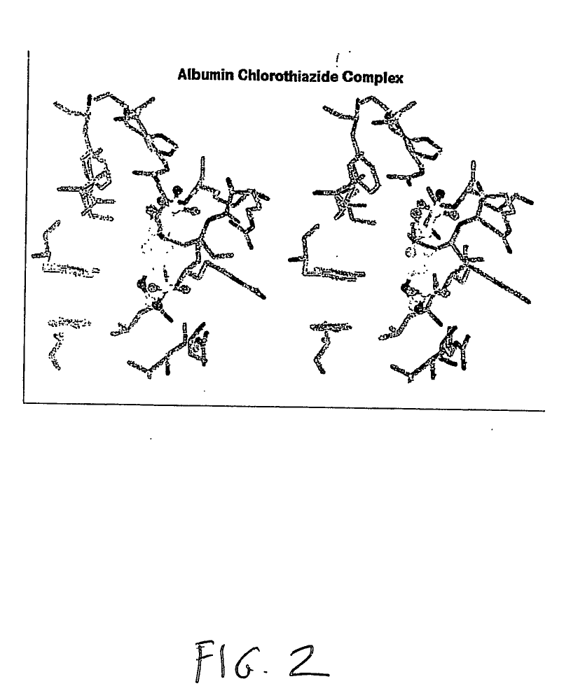

This invention thus provides the structural information showing the binding locations to human serum albumin uniquely associated with each ligand or pharmaceutical (FIG. 1). This information was derived from crystallization of the protein/ligand to create a protein/ligand complex and determining the atomic structure of the resulting complex by x-ray diffraction. The ligand may be any ligand capable of binding to the human serum albumin protein, and is preferably a ligand that binds to one of the binding sites described herein. Examples of such ligands are listed in Table I. and Table II. Preferably, the crystallizable compositions of this invention comprise as the substrate as listed in Tables I and II or a chemical derivative thereof. An important consequence of this extensive body of work, is the recognition of important and totally unappreciated major drug binding regions on the structure of human serum albumin. As indicated above, most notably among these newly discovered interactions has been the identification of the subdomain IB as a major drug binding region (FIG. 2). Additional novel drug binding regions located by this work includes numerous other binding subdomains, including IA, IA/IB, IA/IIA, I/II; I/III; II/III, IIA/IIB, IIB, IIIA/IIIB, IIIB and IIIB, in addition to IB, that can be useful in methods of assessment and in silico prediction. As recognized by one skilled in the art, in silico prediction of drug-binding reactions for use in drug development using computer models is well known, and can be carried out in a number of suitable ways, including but not limited to those models disclosed in Colmenarejo, Medicinal Research Reviews, Vol. 23 (3) 275-301 (2002), incorporated herein by reference. The computer readable databases as set forth above can thus be utilized in silico methods of ADME (Absorption, Distribution, Metabolism and Excretion) assessment of numerous drugs having albumin binding interactions, as would be recognized by one skilled in the art.

In accordance with the invention, the present discoveries have provided new knowledge with regard to the specific binding regions of albumin along with useful information regarding the drug complexes in these regions which can be used to assess and predict drug interactions as would be understood by one skilled in the art. Accordingly, the location of pattern of binding residues allowed by the present invention give insights into the nature of the human serum albumin binding as well as the transport of an incredibly broad class of pharmaceuticals which will be of immense predictive value to the medical and drug development community regarding drug displacement interactions. Still further, the present invention provides a detailed picture of the contacting residues at these sites in a manner not heretofore available so as to allow the development of computer databases and modeling of this information to assess the precise nature and affinity of drug binding to albumin so as to be useful in a variety of drug development activities wherein binding information is needed. Accordingly, the present invention can use the information concerning albumin ligand complexes and coordinates at the contacting binding residues described herein for designing new pharmaceuticals with improved albumin (e.g., increase or decreased binding, shift in albumin binding location) properties.

One method of obtaining information regarding the structural; characteristics of the albumin binding regions is through protein crystallization processes. It has been found that the crystallization of the human serum albumin protein/ligand complexes may be accomplished using a variety of crystallization conditions for each albumin drug complex.

By applying standard crystallization protocols to the above described crystallizable compositions, crystals of the human serum albumin complex may be obtained. This, an even further aspect of this invention relates to a method of preparing human serum albumin complex-containing crystals. The method comprises the steps of
(a) obtaining a crystallizable composition comprising a human serum albumin protein, divalent cations, and a ligand capable of binding to the protein, and
(b) subjecting the composition of step (a) to conditions which promote crystallization.

The conditions for crystallization can include any of those conditions well known in the field by the skilled artisan, or may include such conditions as set forth, e.g., in prior US Patents as indicated in the summary below, each patent incorporated herein by reference:

| | | |
|---|---|---|
| 1 | 5,643,540 | Protein crystal growth apparatus for microgravitiy |
| 2 | 5,641,681 | Device and method for screening crystallization |
| | | conditions in solution crystal growth |
| 3 | 5,585,466 | Crystals of serum albumin for use in genetic engineering |
| | | and rational drug design |
| 4 | 5,419,278 | Vapor equilibration tray for growing protein crystals |
| 5 | 5,130,105 | Protein crystal growth tray assembly |
| 6 | 5,013,531 | Macromolecular crystal growing system |
| 7 | 4,886,646 | Hanging drop crystal growth apparatus and method |
| 8 | 4,833,233 | Human serum albumin crystals and method of |
| | | preparation |
| 9 | 5,780,594 | Biologically active protein fragments containing specific |
| | | binding regions of serum albumin or related proteins |

The structures complexed with the pharmaceuticals or compounds may be readily derived from the amino acids listed in Tables I, II and III. The manner of obtaining these structure coordinates, interpretation of the coordinates and their utility in understanding the protein structure, as described herein, will be understood by those of skill in the art and by reference to standard texts such as Crystal Structure Analysis, Jenny Pickworth Glusker and Kenneth N. Trueblood, 2^{nd} Ed. Oxford University Press, 1985, New York; and Principles of Protein Structure, G. E. Schulz and R. H. Schirmer, Springer-Verlag, 1985, New York.

Those of skill in the art understand that a set of structure coordinates for protein or a protein-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

These variations in coordinates may be generated because of mathematical manipulations of the human serum albumin/ligand structure coordinates. For example, the structure coordinates set forth in Tables I, II, & III could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within an acceptable standard error as compared to the original coordinates, the resulting three-dimensional shape is considered to be the same. This, for example, a ligand that bound to the active site binding pocket of human serum albumin would also be expected to bind to another binding pocket whose structure coordinates defined a shape that fell within the acceptable error.

The term “binding pocket” refers to a region of the protein that, as a result of its shape, favorably associates with a ligand or substrate. The term “serum albumin-like binding pocket” refers to a portion of a molecule or molecular complex whose shape is sufficiently similar to the human serum albumin binding pockets (SABPs) as to bind common ligands as well as pharmaceuticals. This commonality of shape may be quantitatively defined by a root mean square deviation (rmsd) from the structure coordinates of the backbone atoms of the amino acids that make up the SABPs (as set forth in Tables I, II, & III). The method of performing this rmsd calculation is described below.

The “active site binding pockets” or “active site” of human serum albumin refers to one of several areas determined experimentally on the human serum albumin protein surface where substrates bind. In resolving the crystal structure of human serum albumin in complex with ligands, applicants have determined that there exist at least six (7) principle areas of ligand binding on the human serum albumin protein. The sites listed individually in Tables I and II, denote the amino acids which are within 5 Å of and therefore close enough to interact with specific ligand found to bind within this pocket and according to Tables I, II, & III is not more than about 1.15 Å;

These amino acids are hereinafter referred to as the “SET 5 Å amino acids.” Thus, a binding pocket defined by the structural coordinates of those amino acids, as set forth in Tables I, II, & III; or a binding pocket whose root mean square deviation from the structure coordinates of the backbone atoms of those amino acids of not more than about 1.15 angstroms (Å) is considered a serum albumin-like binding pocket of this invention (SABP).

Applicants have also determined that in addition to the human serum albumin amino acids set forth above specific for each Phase II SABP (Table II) produced from refined atomic coordinates of the albumin drug complex, the following residues described in Table I are within 8 Å of bound ligand and therefore are also close enough to interact with that substrate.

These amino acids, in addition to the SET 5A amino acids, are hereinafter referred to as the “SET 8A amino acids.” Thus, in a preferred embodiment, a binding pocket defined by the structural coordinates of the amino acids within 8 Å of bound ligand, as set forth in Tables I, II, & III; or a binding pocket whose root mean square deviation from the structure coordinates of the backbone atoms of those amino acids of not more than about 1.15 Å. is considered a preferred serum albumin-like binding pocket of this invention.

It will be readily apparent to those of skill in the art that the numbering of amino acids in other isoforms of human serum albumin may be different than that set forth for human serum albumin. Corresponding amino acids in other isoforms of human serum albumin are easily identified by visual inspection of the amino acid sequences or by using commercially available homology software programs, as further described below.

Various computational analyses may be used to determine whether a protein or the binding pocket portion thereof is sufficiently similar to the serum albumin binding pockets described above. Such analyses may be carried out in well known software applications, such as the Molecular Similarity application of QUANTA (Molecular Simulations Inc., San Diego, Calif.) version 4.1, and as described in the accompanying User's Guide.

For the purpose of this invention, a rigid fitting method was conveniently used to compare protein structures. Any molecule or molecular complex or binding pocket thereof having a root mean square deviation of conserved residue backbone atoms (N, Calpha., CO) of less than about 1.15 Å when superimposed on the relevant backbone atoms described by structure coordinates listed in Tables I, II, & III are considered identical. More preferably, the root mean square deviation is less than about 1.0 Å.

The human serum albumin X-ray coordinate data, when used in conjunction with a computer programmed with software to translate those coordinates into the 3-dimensional structure of human serum albumin may be used for a variety of purposes, especially for purposes relating to drug discovery. Such software for generating three-dimensional graphical representations are known and commercially available. The ready use of the coordinate data requires that it be stored in a computer-readable format. Thus, in accordance with the present invention, data capable of being displayed as the three dimensional structure of human serum albumin and portions thereof and their structurally similar homologues is stored in a machine-readable storage medium, which is capable of displaying a graphical three-dimensional representation of the structure.

Therefore, another embodiment of this invention provides a machine-readable data storage medium, comprising a data storage material encoded with machine readable data which, when used by a machine programmed with instructions for using said data, displays a graphical three-dimensional representation of a molecule or molecular complex comprising a binding pocket defined by structure coordinates of the human serum albumin SET 5A amino acids, or preferably the human serum albumin SET 8A amino acids, or a homologue of said molecule or molecular complex, wherein said homologue comprises a binding pocket that has a root mean square deviation from the backbone atoms of said amino acids of not more than about 1.15 Å.

Even more preferred is a machine-readable data storage medium that is capable of displaying a graphical three-dimensional representation of a molecule or molecular complex that is defined by the structure coordinates of all of the amino acids in Table III or a homologue of said molecule or molecular complex, wherein said homologue has a root mean square deviation from the backbone atoms of all of the amino acids in Tables I, II, & III of not more than about 1.15 Å.

According to an alternate embodiment, the machine-readable data storage medium comprises a data storage material encoded with a first set of machine readable data which comprises the Fourier transform of the structure coordinates set forth in Tables I, II, & III, and which, when using a machine programmed with instructions for using said data, can be combined with a second set of machine readable data comprising the X-ray diffraction pattern of another molecule or molecular complex to determine at least a portion of the structure coordinates corresponding to the second set of machine readable data.

For example, the Fourier transform of the structure coordinates set forth in Tables I, II, & III may be used to determine at least a portion of the structure coordinates of other serum albumins. The structure coordinates derived from Tables I, II, & III and the Fourier transform of the coordinates of refined albumin complexes are especially useful for determining the coordinates of other albumins in ligand/complex form.

According to an alternate embodiment, this invention provides a computer for producing a three-dimensional representation of a molecule or molecular complex, wherein said molecule or molecular complex comprises a binding pocket defined by the human serum albumin SET 5A amino acids, or preferably the human serum albumin SET 8A amino acids, or a homologue of said molecule or molecular complex, wherein said homologue comprises a binding pocket that has a root mean square deviation from the backbone atoms of said amino acids of not more than 1.15 Å wherein said computer comprises:
(a) a machine readable data storage medium comprising a data storage material encoded with machine-readable data, wherein said machine readable data comprises the structure coordinates of human serum albumin or portions thereof;
(b) a working memory for storing instructions for processing said machine-readable data;
(c) a central-processing unit coupled to said working memory and to said machine-readable data storage medium, for processing said machine-readable data into said three-dimensional representation; and
(d) an output hardware coupled to said central processing unit, for receiving said three Dimensional representation.

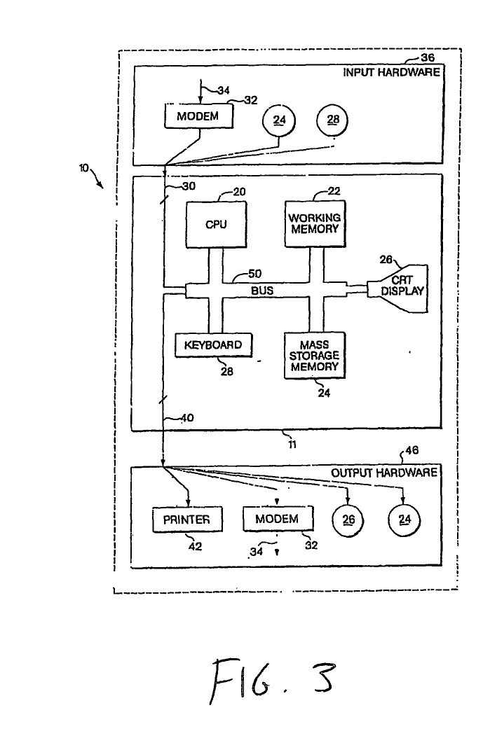

FIG. 3 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bi-directional system bus 50.

Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding pocket of this invention using a program such as QUANTA as described herein. Output hardware might also include a printer 42, so that hard copy output may be produced, or a disk drive 24, to store system output for later use.

In operation, CPU 20 coordinates the use of the various input and output devices 36, 46 coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

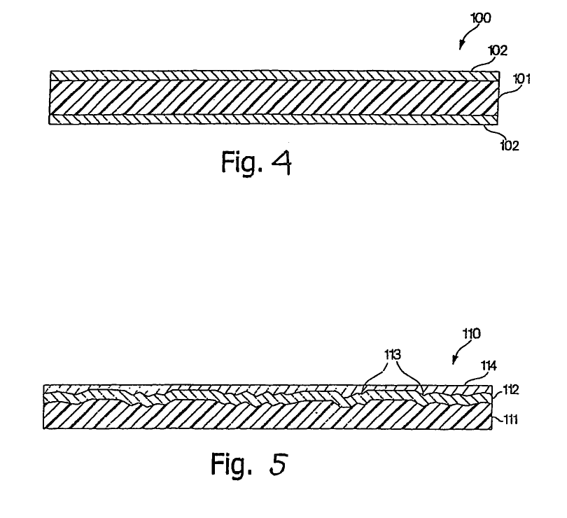

FIG. 4 shows a cross section of a magnetic data storage medium 100 which can be encoded with a machine-readable data that can be carried out by a system such as system 10 of FIG. 8. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24. The magnetic domains of coating 102 of medium 100 are polarized or oriented so as to encode in manner which may be conventional, machine readable data such as that described herein, for execution by a system such as system 10 of FIG. 3.

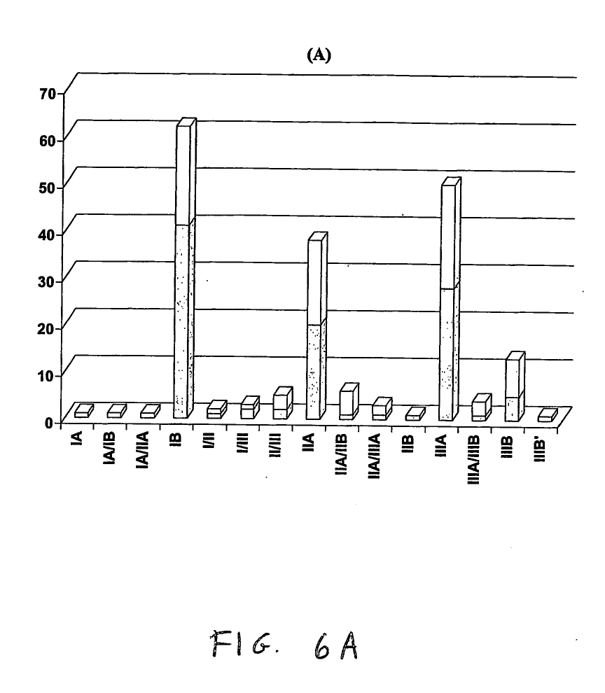

FIG. 5 shows a cross section of an optically-readable data storage medium 110 which also can be encoded with such a machine-readable data, or set of instructions, which can be carried out by a system such as system 10 of FIG. 3. Medium 110 can be a conventional compact disk read only memory (CD-ROM) or a rewritable medium such as a magneto-optical disk which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

As mentioned above, the human serum albumin X-ray coordinate data is useful for screening and identifying drugs that are bound by serum albumin, especially those listed in Tables I and II. For example, the structure encoded by the data may be computationally evaluated for its ability to associate with putative substrates or ligands. Such compounds that associate with human serum albumin are useful in the design or recognition of potential drug candidates. Additionally or alternatively, the structure encoded by the data may be displayed in a graphical three-dimensional representation on a computer screen. This allows visual inspection of the structure, as well as visual inspection of the structure's association with the compounds.

Thus, according to another embodiment, this invention relates to a method for evaluating the potential of a compound to associate with a molecule or molecular complex, comprising a binding pocket defined by the structure coordinates of the human serum albumin SET 5A amino acids, or preferably the human serum albumin SET 8A amino acids, or a homologue of said molecule or molecular complex, wherein said homologue comprises a binding pocket that has a root mean square deviation from the backbone atoms of said amino acids of not more than about 1.15 Å.

This method comprises the steps of:
(a) creating a computer model of the binding pocket using structure coordinates wherein the root mean square deviation between said structure coordinates and the structure coordinates of the human serum albumin amino acids outlined in Tables I, II, & III is not more than about 1.15 Å;
(b) employing computational means to perform a fitting operation between the chemical entity and said computer model of the binding pocket; and
(c) analyzing the results of said fitting operation to quantify the association between the chemical entity and the binding pocket model.

The term “chemical entity”, as used herein, refers to chemical compounds or ligands, complexes of at least two chemical compounds, and fragments of such compounds or complexes.

Even more preferably, the method evaluates the potential of a chemical entity to associate with a molecule or molecular complex defined by the structure coordinates of all of the human serum albumin amino acids, as set forth in Tables I, II, & III, or a homologue of said molecule or molecular complex having a root mean square deviation from the backbone atoms of said amino acids of not more than 1.15 Å.

Alternatively, the structural coordinates of the human serum albumin binding pocket can be utilized in a method for identifying a potential agonist or antagonist of a molecule comprising a serum albumin-like binding pocket. This method comprises the steps of:
(a) using atomic coordinates of the human serum albumin SET 5A amino acids .+−. a root mean square deviation from the backbone atoms of said amino acids of not more than about 1.15 Å, to generate a three-dimensional structure of molecule comprising a serum albumin-like binding pocket;
(b) employing said three-dimensional structure to design or select said potential agonist or antagonist;
(c) synthesizing said agonist or antagonist; and
(d) contacting said agonist or antagonist with said molecule to determine the ability of said potential agonist or antagonist to interact with said molecule.

More preferred is the use of the atomic coordinates of the human serum albumin SET 8A amino acids, ± a root mean square deviation from the backbone atoms of said amino acids of not more than 1.15 Å, to generate a three-dimensional structure of molecule comprising a SABP. Most preferred is when the atomic coordinates of all the amino acids of human serum albumin according to Tables I, II, & III ± a root mean square deviation from the backbone atoms of said amino acids of not more than 1.15 Å, are used to generate a three-dimensional structure of molecule comprising a SABP.

The present invention permits the use of molecular design techniques to identify, select or design potential pharmaceutical interacting with human serum albumin, based on the structure of a ligand complexed with a serum albumin-like binding pocket. Such a predictive model is valuable in light of the high costs associated with the preparation and testing of the many diverse compounds that may possibly bind to the serum albumin protein.

According to this invention, a potential serum albumin ligand may now be evaluated for its ability to bind a serum albumin-like binding pocket prior to its actual synthesis and testing. If a proposed compound is predicted to have undesired interaction or association with the binding pocket, preparation and testing of the compound is obviated. However, if the computer modeling indicates properties with desirable interaction, the compound may then be obtained and tested for its ability to bind. Testing to confirm binding may be performed using methods such as microcalorimetery, equilibrium dialysis, or surface plasmon resonance.

A potential ligand bound to a serum albumin-like binding pocket may be computationally evaluated by means of a series of steps in which chemical entities or fragments are screened and selected for their ability to associate with the serum albumin-like binding pockets.

One skilled in the art may use one of several methods to screen chemical entities or fragments for their ability to associate with a human serum albumin-like binding pocket. This process may begin by visual inspection of, for example, a human serum albumin-like binding pocket on the computer screen based on the human serum albumin structure coordinates in Tables I, II, & III or other coordinates which define a similar shape generated from the machine-readable storage medium. Selected fragments or chemical entities may then be positioned in a variety of orientations, or docked, within that binding pocket as defined above. Docking may be accomplished using software such as Quanta and Sybyl, followed by energy minimization and molecular dynamics with standard molecular mechanics force fields, such as CHARMM and AMBER.

Specialized computer programs may also assist in the process of selecting fragments or chemical entities. These include:
1. GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK.
2. MCSS (A. Miranker it al., “Functionality Maps of Binding Sites: A Multiple Copy Simultaneous Search Method.” Proteins: Structure, Function and Genetics, 11, pp. 29-34 (1991)). MCSS is available from Molecular Simulations, San Diego, Calif.
3. AUTODOCK (D. S. Goodsell et al., “Automated Docking Substrates to Proteins by Simulated Annealing”, Proteins: Structure, Function, and Genetics, 8, pp. 195-202 (1990)). AUTODOCK is available from Scripps Research Institute, La Jolla, Calif.
4. DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif.

Once suitable chemical entities or fragments have been selected, they can be designed or assembled into a single compound or complex. Assembly may be preceded by visual inspection of the relationship of the fragments to each other on the three-dimensional image displayed on a computer screen in relation to the structure coordinates of human serum albumin. This would be followed by manual model building using software such as Quanta or Sybyl [Tripos Associates, St. Louis, Mo.].

Useful programs to aid one of skill in the art in connecting the individual chemical entities or fragments include:
1. CAVEAT (P. A. Bartlett et al, “CAVEAT: A Program to Facilitate the Structure-Derived Design of Biologically Active Molecules”, in Molecular Recognition in Chemical and Biological Problems”, Special Pub., Royal Chem. Soc., 78, pp. 182-196 (1989); G. Lauri and P. A. Bartlett, “CAVEAT: a Program to Facilitate the Design of Organic Molecules”, J. Comput Aided Mol. Des., 8, pp. 51-66 (1994)). CAVEAT is available from the University of California, Berkeley, Calif.
2. 3D Database systems such as ISIS (MDL Information Systems, San Leandro, Calif.). This area is reviewed in Y. C. Martin. “3D Database Searching in Drug Design”, J. Med. Chem., 35, pp. 2145-2154 (1992).
3. HOOK (M. B. Eisen et al, “HOOK: A Program for Finding Novel Molecular Architectures that Satisfy the Chemical and Steric Requirements of a Macromolecule Binding Site”, Proteins: Struct., Funct. Genet., 19, pp. 199-221 (1994). HOOK is available from Molecular Simulations, San Diego, Calif.

Instead of proceeding to build an inhibitor or drug with reduced binding to a human serum albumin-like binding pocket in a step-wise fashion one fragment or chemical entity at a time as described above, inhibitory or other human serum albumin binding compounds may be designed as a whole or “de novo” using either an empty binding site or optionally including some portion(s) of a known inhibitor(s). There are many de novo ligand design methods including:
1. LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.
2. LEGEND (Y. Nishibata et al., Tetrahedron, 47, p. 8985 (1991)). LEGEND is available from Molecular Simulations Incorporated, San Diego, Calif.
3. LeapFrog (available from Tripos Associates, St. Louis, Mo.).
4. SPROUT (V. Gillet et al, “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention [see, e.g., Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)].

Once a compound has been designed or selected by the above methods, the efficiency with which that entity may bind to a SABP may be tested and optimized by computational evaluation

An entity designed or selected as binding to a SABP may be further computationally optimized so as (for example) to reduce its affinity to a specific SABP, the difference in efficiency with which that entity may bind (or not bind) may be tested computationally.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. .COPYRGT.1995); AMBER, version 4.1 (P. A. Kollman, University of California at San Francisco,.COPYRGT.1995) QUANTA/CHARMM (Molecular Simulations, Inc., San Diego, Calif. .COPYRGT.1995); Insight II/Discover (Molecular Simulations, Inc., San Diego, Calif. .COPYRGT.1995); DelPhi (Molecular Simulations, Inc., San Diego, Calif. .COPYRGT.1995); and AMSOL (Qunatum Chemistry Program Exchange, Indiana University). These programs may be implemented, for instance, using a Silicon Graphics workstation such as an Indigo.sup.2 with “IMPACT” graphics. Other hardware systems and software packages will be known to those skilled in the art.

Another approach enabled by this invention, is the computational screening of small molecule databases for chemical entities or compounds that can bind in whole, or in part, to a human serum albumin binding pocket. In this screening, the quality of fit of such entities to the binding site may be judged either by shape complementarity or by estimated interaction energy [E. C. Meng et al., J. Comp. Chem., 13, 505-524 (1992)].

According to another embodiment, the invention provides compounds (such as those listed in Tables I & II which associate with a human serum albumin-like binding pocket, and which may be further expanded upon by ab initio methods produced or identified by the method set forth above.

The structure coordinates set forth in Tables I, II, & III can also be used to aid in obtaining structural information about another crystallized molecule or molecular complex. This may be achieved by any of a number of well-known techniques, including molecular replacement.

Therefore, in another embodiment this invention provides a method of utilizing molecular replacement to obtain structural information about a molecule or molecular complex whose structure is unknown comprising the steps of:
a. crystallizing said molecule or molecular complex of unknown structure;
b. generating an X-ray diffraction pattern from said crystallized molecule or molecular complex; and
c. applying at least a portion of the structure coordinates set forth in Tables I, II, & III to the X-ray diffraction pattern to generate a three-dimensional electron density map of the molecule or molecular complex whose structure in unknown.

By using molecular replacement, all or part of the structure coordinates of the human serum albumin complex as provided by this invention (and set forth in Tables I, II, & III) can be used to determine the structure of another crystallized molecule or molecular complex more quickly and efficiently than attempting an ab initio structure determination.

Molecular replacement provides an accurate estimation of the phases for an unknown structure. Phases are a factor in equations used to solve crystal structures that can not be determined directly. Obtaining accurate values for the phases, by methods other than molecular replacement, Is a time-consuming process that involves interactive cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of the human serum albumin complex according to Tables I, II, & III within the unit cell of the crystal of the unknown molecule or molecular complex so as best to account for the observed X-ray diffraction pattern of the crystal of the molecule or molecular complex whose structure in unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex [E. Lattmen, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)].

The structure of any portion of any crystallized molecule or molecular complex that is sufficiently homologous to any portion of the human serum albumin/ligand complex can be resolved by this method.

In a preferred embodiment, the method of molecular replacement is utilized to obtain structural information about other serum albumins, such as mouse, rat dog, rabbit, etc, as may be useful in drug development or isoforms of human serum albumin. The structure coordinates of human serum albumin as provided by this invention are particularly useful in solving the structure of other isoforms of human serum albumin, other members of the serum albumin family of proteins, including vitamin D-binding protein, alpha-fetoprotein, or human serum albumin complexes.

Furthermore, the structure coordinates of human serum albumin as provided by this invention are useful in solving the structure of human serum albumin proteins that have amino acid substitutions, additions and/or deletions (referred to collectively as “human serum albumin mutants,” as compared to naturally occurring human serum albumin isoforms). These human serum albumin mutants may optionally be crystallized in co-complex with a chemical entity, such as a analogue or a suicide substrate. The crystal structures of a series of such complexes may then be solved by molecular replacement and compared with that of wild-type human serum albumin. Potential sites for modification within the various binding sites of the enzyme may thus be identified. This information provides an additional tool for determining the most efficient binding interactions such as, for example, increasing or decreasing hydrophobic interactions, between human serum albumin and a chemical entity or compound.

All of the complexes referred to above may be studied using well-known X-ray diffraction techniques and may be refined versus 1.5-3 Å resolution X-ray data to an R value of about 0.22 or less using computer software, such as X-PLOR [Yale University, COPYRIGHT 1992, distributed by Molecular Simulations, Inc.; see, e.g., Blundell & Johnson, supra; Meth. Enzymol., vol. 114 & 115, H. W. Wyckoff et al., eds., Academic Press (1985)1. This information may thus be used to optimize known human serum albumin bound pharmaceuticals, and more importantly, to design improved pharmaceuticals with improved binding properties to human serum albumin.

The structure coordinates described above may also be used to derive the dihedral angles, phi. and psi., that define the conformation of the amino acids in the protein backbone. As will be understood by those skilled in the art, the phi. sub n angle refers to the rotation around the bond between the alpha carbon and the nitrogen, and the phi..sub.n angle refers to the rotation around the bond between the carbonyl carbon and the alpha carbon. The subscript “n” identifies the amino acid whose conformation is being described [for a general reference, see Blundell and Johnson, Protein Crystallography, Academic Press, London,

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Examples

### Example 1

### Method of Determining Contacting Residues for Albumin Binding Regions

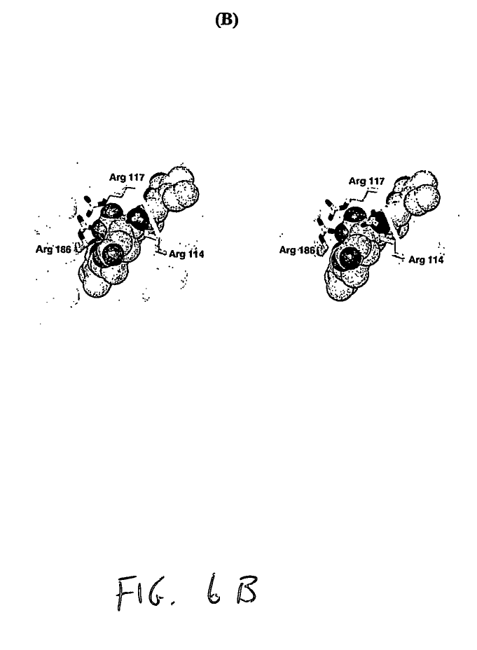

The compounds for this study were selected from more than 1000 clinically approved pharmaceuticals based on high plasma binding and/or high affinity to HSA. This approach resulted in an initial list of 350 targeted pharmaceuticals and a few selected drug-like molecules of interest. The paucity of 3-dimensional albumin drug binding data in the literature is a direct testimony to the difficulty in experimental processes due to albumin's inherent conformational flexibility. However, using our proprietary CADEX™ technology, we have, so far, resolved more than 140 structures representative of every major therapeutic indication, providing for an unprecedented view of albumin drug binding chemistry (FIG. 6A, Table III).

Contemporary studies of HSA drug-binding have been greatly influenced by the early work of Sudlow et al., who identified two dominant binding locations by equilibrium dialysis methods, denoted Site I and II (1). Our early crystallographic studies using a small sampling of compounds identified two major sites in subdomains IIA and IIIA which correlated with Sudlow's Site I and II, respectively (2,3). However, this survey indicates that the two-site description is oversimplified and inaccurate for HSA drug interactions. Consequently, it is more appropriate to use an unambiguous nomenclature system referencing subdomain locations as outlined in FIG. 6A and Table III.

In all, 15 independent binding locations have presently been identified by this survey (FIG. 6A). Of the 15, three principal sites dominate, accounting for >80% of drug binding locations currently determined. More than 70% (105) of the complexes reveal drugs bound to discrete single occupancy sites, twenty four drugs show two sites and thirteen have more than two binding locations. Eight compounds exhibited preferential binding of two molecules within a single site. Of the single occupancy complexes, 39% are located within Site IB, 19% within Site IIA and 27% within Site IIIA (FIG. 6A). Impressively, 44% of all compounds surveyed in this study have at least one-binding site at subdomain IB.

The structural details of the subdomain IB site are illustrated in FIG. 6B. Previously this location has been identified with endogenous ligand binding such as long-chain fatty acids and heme (4). This binding region, the major drug binding site on HSA, has the largest capacity for accommodating ligands, e.g., complex heterocyclic compounds. For instance, bilirubin, a Sudlow Site I marker and toxic heme metabolite, is considered to be one of the most extensively studied ligand interactions with albumin (5). This survey located bilirubin at subdomain IB, instead of the presumed IIA site (FIG. 6B). This location explains the reduced affinity for bilirubin observed for the HSA variant Yanomama-2 (114R→G) (6).

In summary, the more accurate view of albumin binding chemistry pursuant to the discoveries in association with the present invention, together with the illumination of the principal albumin ligand binding site, brings clarity to an immense body of conflicting results in the literature. The high frequency in single site drug complexes observed underscores the applicability of the structural information to improve the safety and efficacy of many existing and newly developing pharmaceuticals. Furthermore, the extensive data accumulated to date offers great potential for predictive capabilities in drug binding, drug displacement interactions and in silico ADME.

TABLE 3
Location, Frequency and Description of Human Serum Albumin Drug Binding Sites (Sequence shown,
e.g., in U.S. Pat. No. 5,780,594, incorporated herein by reference)

| Drug | | Single Site | |
|---|---|---|---|
| Binding | Frequency | Frequency | |
| Site | (%) | (%) | Residues Surrounding The Sites |
| IA | 1(0.52%) | 1(0.95%) | V007, F019, V023, F027, E045, V046, F049, A050, E060, N061, |
| | | | K064, L066, L069, F070, G071, D072, K073, C075, T076, C091, |
| | | | R098, L251 |
| IA/IB | 1(0.52%) | 0(0.00%) | E017, N018, A021, E132, L135, L139, L155, A158, K159 |
| IA/IIA | 1(0.52%) | 0(0.00%) | V007, F019, V023, A026, V046, F049, L066, H067, L069, F070, |
| | | | G248, D249, L250, L251, E252 |
| IB | 62(32.46%) | 41(39.05%) | F036, F037, D108, P110, N111, L112, P113, R114, L115, V116, |
| | | | R117, P118, V122, M123, A126, N130, T133, F134, L135, K137, |
| | | | Y138, L139, Y140, E141, I142, A143, R145, H146, P147, Y148, |
| | | | F149, Y150, L154, F157, A158, Y161, F165, L182, D183, L185, |
| | | | R186, D187, G189, K190, K190, S192, S193, A194, Q196, R197, |
| | | | E425, Q459 |
| I/II | 2(1.05%) | 1(0.95%) | E100, L103, Q104, D108, R145, H146, P147, Y148, F149, S193, |
| | | | Q196, R197, C200, A201, Q204, K205, C245, C246, H247, G248, |
| | | | N458, V462 |
| I/III | 3(1.57%) | 2(1.90%) | D108, N109, R145, H146, R186, D187, E188, K190, A191, S193, |
| | | | A194, R197, P421, T422, E425, R428, N429, K432, V433, K436, |
| | | | Y452, V455, V456, Q459, V462, L463, K519, I523 |
| II/III | 5(0.62%) | 2(1.90%) | A194, K195, R197, L198, K199, C200, A201, S202, L203, K205, |
| | | | F206, G207, A210, F211, A213, W214, H242, C246, V343, V344, |
| | | | L347, E450, D451, L453, V455, S454, L457, N458, C461, V462, |
| | | | E465, C477, T478, E479, S480, L481, V482, R484, R485 |
| IIA | 38(19.90%) | 20(19.05%) | F149, Y150, E153, A191, S192, K195, Q196, L198, K199, C200, |
| | | | S202, F211, W214, A215, R218, L219, R222, F223, L234, L238, |
| | | | V241, H242, C245, C246, C253, D256, R257, L260, A261, I264, |
| | | | K286, S287, H288, I290, A291, E292, V293, V343, P447, D451, |
| | | | Y452, V455 |
| IIA/IIB | 6(3.14%) | 1(0.95%) | L198, K199, S202, F206, R209, A210, F211, K212, A213, W214, |
| | | | V216, F228, V231, S232, D324, V325, L327, G328, L331, V343, |
| | | | V344, L347, A350, K351, E354, D451, S454, E479, S480, L481, |
| | | | V482, N483 |
| IIB | 1(0.52%) | 1(0.95%) | D308, F309, N318, E321, A322, V325, F326, M329 |
| IIIA | 50(26.18%) | 28(26.67%) | E383, P384, K387, L387, I388, Q390, A391, N391, C392, F395, |
| | | | F403, L407, L408, R410, Y411, K414, V415, V418, T422, L423, |
| | | | V424, V426, S427, L430, G431, V433, G434, S435, C437, C438, |
| | | | R445, M446, A449, E450, W450, Y452, L453, V456, L457, L460, |
| | | | V473, R484, R485, F488, S489, A490, L491, W492 |
| IIIA/IIIB | 4(2.09%) | 1(0.95%) | L398, Y401, K402, N405, A406, L407, V409, R410, K413, K525, |
| | | | L529, A539, T540, K541, E542, L544, K545, M548, D549, A552 |
| IIIB | 13(6.81%) | 5(4.76%) | Y401, K402, N405, F502, F507, F509, I513, L516, R521, K524, |
| | | | K525, Q526, A528, L529, E531, L532, V533, H535, K536, A539, |
| | | | T540, Q543, L544, K545, V547, M548, D549, D550, F551, A552, |
| | | | F554, V555, E556, G572, L575, V576, A577, A578, S579 |
| IIIB′ | 1(0.52%) | 1(0.95%) | C514, E518, R521, V555, E556, C559, K560 |

(/) indicates binding at the interface between two domains or subdomains. The numbers in the table were derived from 142 complex structures determined so far. There are 105 single-site complexes. The remaining 37 show multiple binding locations.

### APPENDIX A:

The following patent and journal references are incorporated into the present specification by reference as if set forth in their entirety herein

### JOURNAL REFERENCES

1. G. Sudlow, D. J. Birkett, and D. N. Wade, Mol. Pharmacol. 12,1052 (1975).
2. X. M. He and D. C. Carter, Nature, 358, 209 (1992).
3. D. C. Carter and J. X. Ho, in Adv. Protein Chem. (V. Shoemaker, ed.) 45, (1994), 153-204.
4. M. Wardell, et al., Biochem. Biophys. Res. Com., 291, 813 (2002).
5. R. Brodersen, Crit. Rev. Clin. Lab. Sci., 11, 305 (1980).
6. N. Takahashi, et al., Proc. Natl. Acad. Sci. U.S.A., 84, 8001 (1987).

### PATENT REFERENCES

| | | |
|---|---|---|
| US-5,322,933 | Jun. 21, 1994 | DAVIES et al. |
| US-5,882,878 | Mar. 16, 1999 | SACCHETTINI |
| US-5,939,528 | Aug. 17, 1999 | CLARDY et al. |
| US-6,057,119 | May 2, 2000 | WILSON et al. |
| US-6,093,573 | Jul. 25, 2000 | BEAMER et al. |
| US-6,162,613 | Dec. 19, 2000 | SU et al. |
| US-6,387,641 | May 14, 2002 | BELLON et al. |
| US-6,459,996 | Oct. 1, 2002 | SOMERS et al. |
| US- | Apr. 29, 2004 | |
| 20040082052A1 | | |
| US- | Jan. 15, 2004 | |
| 20040009568A1 | | |
| US- | Jan. 8, 2004 | |
| 20040005686A1 | | |
| US- | Dec. 4, 2003 | |
| 20030229453A1 | | |
| US- | Dec. 11, 2003 | |
| 20030229453A1 | | |
| US- | Dec. 4, 2003 | |
| 20030224500A1 | | |
| US- | Dec. 4, 2003 | |
| 20030224368A1 | | |
| US- | Dec. 4, 2003 | |
| 20030224006A1 | | |
| US- | Oct. 23, 2003 | |
| 20030198997A1 | | |
| US- | Oct. 2, 2003 | |
| 20030187220A1 | | |
| US- | Sep. 11, 2003 | |
| 20030171904A1 | | |
| US- | Sep. 11, 2003 | |
| 20030171549A1 | | |
| US- | Sep. 11, 2003 | |
| 20030170868A1 | | |
| US- | Sep. 4, 2003 | |
| 20030166843A1 | | |
| US- | Sep. 4, 2003 | |
| 20030166233A1 | | |
| US- | Sep. 4, 2003 | |
| 20030165984A1 | | |
| US- | Aug. 21, 2003 | |
| 20030158384A1 | | |
| US- | Jul. 31, 2003 | |
| 20030143714A1 | | |
| US- | Jul. 10, 2003 | PARK et al. |
| 20030129656A1 | | |
| US- | Jul. 3, 2003 | |
| 20030125332A1 | | |
| US- | Jun. 5, 2003 | |
| 20030105010A1 | | |
| US- | May 29, 2003 | |
| 20030101005A1 | | |
| US- | May 22, 2003 | |
| 20030096363A1 | | |
| US- | May 1, 2003 | |
| 20030082769A1 | | |
| US- | Apr. 17, 2003 | |
| 20030073134A1 | | |
| US- | Feb. 13, 2003 | |
| 20030032779A1 | | |
| US- | Jan. 23, 2003 | |
| 20030018166A1 | | |
| US- | Nov. 21, 2002 | |
| 20020172670A1 | | |
| US- | Nov. 7, 2002 | |
| 20020165155A1 | | |
| US- | Oct. 24, 2002 | |
| 20020156585A1 | | |
| US- | Sep. 19, 2002 | |
| 20020132319A1 | | |
| US- | Jun. 13, 2002 | BENSON et al. |
| 20020072105A1 | | |
| US- | Mar. 21, 2002 | |
| 20020034802A1 | | |
| US- | | |
| 20020031782A1 | | |
| US- | Feb. 13, 2003 | GOLDSMITH et al. |
| 20030032649A1 | | |

What is claimed is:

1. A method for evaluating the ability of a compound to associate with a molecule or molecular complex comprising a human serum albumin binding region selected from the group consisting of binding subdomains IA, IA/IB, IA/IIA, IB, I/II; I/III; II/III, IIA, IIA/IIB, IIB, IIIA, IIIA/IIIB, IIIB and IIIB′, said method comprising the steps of:
a) constructing a computer model of said binding region defined by three-dimensional structural binding coordinates wherein the root mean square deviation between said structural binding coordinates and the structure binding coordinates of the respective binding region as set forth in Table S1 is not more than about 1.15 angstroms;
b) selecting a compound to be evaluated by a method selected from the group consisting of (i) assembling molecular fragments into said compound, (ii) selecting a compound from a small molecule database, (iii) de novo ligand design of said compound, (iv) a compound obtained by modifying a compound with known binding affinity to a human serum albumin binding region; (v) a pharmaceutical or other compound as set forth in Tables I or II; (vi) a compound obtained by modifying a known pharmaceutical compound, or active portion thereof, of human serum albumin
c) employing computational means to perform a fitting program operation between computer models of the said compound to be evaluated and said binding region in order to provide an energy-minimized configuration of the said compound in the binding region; and
d) evaluating the results of said fitting operation to quantify the association between the said compound and the binding region computer model, thereby evaluating the ability of the said compound to associate with the said binding region.

2. The method of claim 1 wherein the root mean square deviation is within about 2.5 angstroms.

3. The method of claim 1 wherein the root mean square deviation is within about 3.0 angstroms.

4. The method of claim 1 wherein the psi angle is in the range of about −30 degrees. to +30 degrees

5. The method of claim 1 wherein the .phi. angle is in the range of about 60 degrees to 120.degrees.

6. The method of claim 1 wherein the human serum albumin binding subdomain is binding region 1B, and wherein the three-dimensional structural binding coordinates at positions F036, F037, D108, P110, N111, L112, P113, R114, L115, V116, R117, P118, V122, M123, A126, N130, T133, F134, L135, K137, Y138, L139, Y140, E141, I142, A143, R145, H146, P147, Y148, F149, Y150, L154, F157, A158, Y161, F165, L182, D183, L185, R186, D187, G189, K190, K190, S192, S193, A194, Q196, R197, E425, Q459, and a root mean square deviation from the backbone atoms of said amino acids of not more than 1.15 angstroms, are used to generate said three-dimensional structure of said binding region defined by three-dimensional structural binding coordinates.

7. The method of claim 6 wherein the compound to be evaluated is a compound binding to the IB subdomain having three dimensional structural coordinates at the positions shown in Tables 1 and 2

8. The method of claim 1 wherein the human serum albumin binding subdomain is binding region IIA, and wherein the three-dimensional structural binding coordinates at positions F149, Y150, E153, A191, S192, K195, Q196, L198, K199, C200, S202, F211, W214, A215, R218, L219, R222, F223, L234, L238, V241, H242, C245, C246, C253, D256, R257, L260, A261, 1264, K286, S287, H288, I290, A291, E292, V293, V343, P447, D451, Y452, V455, and a root mean square deviation from the backbone atoms of said amino acids of not more than 1.15 angstroms are used to generate said three-dimensional structure of said binding region defined by three-dimensional structural binding coordinates.

9. The method of claim 8 wherein the compound to be evaluated is a compound binding to the IIA subdomain having three dimensional structural coordinates at the positions as shown in Tables 1 and 2

10. The method of claim 1 wherein the human serum albumin binding subdomain is binding region IIIA, and wherein the three-dimensional structural binding coordinates at positions E383, P384, K387, L387, I388, Q390, A391, N391, C392, F395, F403, L407, L408, R410, Y411, K414, V415, V418, T422, L423, V424, V426, S427, L430, G431, V433, G434, S435, C437, C438, R445, M446, A449, E450, W450, Y452, L453, V456, L457, L460, V473, R484, R485, F488, S489, A490, L491, W492, and a root mean square deviation from the backbone atoms of said amino acids of not more than 1.15 angstroms are used to generate said three-dimensional structure of said binding region defined by three-dimensional structural binding coordinates.

12. The method of claim 10 wherein the compound to be evaluated is a compound binding to the IIIA subdomain having three dimensional structural coordinates at the positions shown in Tables 1 and 2

13. The method of claim 1 wherein the human serum albumin binding subdomain is binding region IA, and wherein the three-dimensional structural binding coordinates at positions V007, F019, V023, F027, E045, V046, F049, A050, E060, N061, K064, L066, L069, F070, G071, D072, K073, C075, T076, C091, R098, L251, and a root mean square deviation from the backbone atoms of said amino acids of not more than 1.15 angstroms are used to generate said three-dimensional structure of said binding region defined by three-dimensional structural binding coordinates.

14. The method of claim 13 wherein the compound to be evaluated is a compound binding to the IA subdomain having three dimensional structural coordinates at the positions shown in Tables 1 and 2.

15. The method of claim 1 wherein the human serum albumin binding subdomain is binding region IIA-IIB, and wherein the three-dimensional structural binding coordinates at positions L198, K199, S202, F206, R209, A210, F211, K212, A213, W214, V216, F228, V231, S232, D324, V325, L327, G328, L331, V343, V344, L347, A350, K351, E354, D451, S454, E479, S480, L481, V482, N483 and a root mean square deviation from the backbone atoms of said amino acids of not more than 1.15 angstroms are used to generate said three-dimensional structure of said binding region defined by three-dimensional structural binding coordinates.

16. A method for identifying an activator or inhibitor of a molecule comprising a human serum albumin binding region selected from the group consisting of binding region IA, IA/IB, IA/IIA, IB, I/II; I/III; II/III, IIA, IIA/IIB, IIB, IIIA, IIIA/IIIB, IIIB and IIIB′ comprising the steps of
a) constructing a computer model of the binding region defined by three-dimensional structural binding coordinates wherein the root mean square deviation between said structural binding coordinates and the structure binding coordinates of the respective binding region as set forth in TableS1 is not more than about 1.15 angstroms;
b) selecting a compound to be evaluated by a method selected from the group consisting of (i) assembling molecular fragments into said compound, (ii) selecting a compound from a small molecule database, (iii) de novo ligand design of said compound, (iv) a compound obtained by modifying a compound with known binding affinity to a human serum albumin binding region; (v) a pharmaceutical or other compound as set forth in Tables I or II; (vi) a compound obtained by modifying a known pharmaceutical compound, or active portion thereof, of human serum albumin
c) employing computational means to perform a fitting program operation between computer models of the said compound to be evaluated and said binding region in order to provide an energy-minimized configuration of the said compound in the binding region;
d) evaluating the results of said fitting operation to quantify the association between the said compound and the binding region computer model, thereby evaluating the ability of the said compound to associate with the said binding region;
e) synthesizing said compound; and
f) contacting said compound with said molecule to determine the ability of said compound to activate or inhibit said molecule.

17. A method for identifying a ligand interaction with a molecule or molecule complex comprising a human serum albumin binding region selected from the group consisting of binding region IA, IA/IB, IA/IIA, IB, I/II; I/III; II/III, IIA; IIA/IIB, IIB, IIIA, IIIA/IIIB, IIIB and IIIB′ comprising the steps of
a) constructing a computer model of the binding region defined by three-dimensional structural binding coordinates wherein the root mean square deviation between said structural binding coordinates and the structure binding coordinates of the respective binding region as set forth in TableS1 is not more than about 1.15 angstroms;
b) selecting a compound to be evaluated by a method selected from the group consisting of (i) assembling molecular fragments into said compound, (ii) selecting a compound from a small molecule database, (iii) de novo ligand design of said compound, (iv) a compound obtained by modifying a compound with known binding affinity to a human serum albumin binding region; (v) a pharmaceutical or other compound as set forth in Tables I or II; (vi) a compound obtained by modifying a known pharmaceutical compound, or active portion thereof, of human serum albumin
c) employing computational means to perform a fitting program operation between computer models of the said compound to be evaluated and said binding region in order to provide an energy-minimized configuration of the said compound in the binding region;
d) evaluating the results of said fitting operation to quantify the association between the said compound and the binding region computer model, thereby evaluating the ability of the said compound to associate with the said binding region;
e) synthesizing said compound; and
f) contacting said compound with said molecule so as to determine the ability of said ligand interact with said molecule.

18. The method of claim 17 wherein the root mean square deviation is within about 2.5 angstroms.

19. The method of claim 17 wherein the root mean square deviation is within about 3.0 angstroms.

20. A method of optimizing the binding of a compound to a human serum albumin comprising a human serum albumin binding region selected from the group consisting of binding region IA, IA/IB, IA/IIA, IB, I/II; I/III; II/III, IIA, IIA/IIB, IIB, IIIA, IIIA/IIIB, IIIB and IIIB′ comprising the steps of
a) constructing a computer model of the binding region defined by three-dimensional structural binding coordinates wherein the root mean square deviation between said structural binding coordinates and the structure binding coordinates of the respective binding region as set forth in TableS1 is not more than about 1.15 angstroms;
b) selecting a compound to be evaluated by a method selected from the group consisting of (i) assembling molecular fragments into said compound, (ii) selecting a compound from a small molecule database, (iii) de novo ligand design of said compound, (iv) a compound obtained by modifying a compound with known binding affinity to a human serum albumin binding region; (v) a pharmaceutical or other compound as set forth in Tables I or II; (vi) a compound obtained by modifying a known pharmaceutical compound, or active portion thereof, of human serum albumin
c) employing computational means to perform a fitting program operation between computer models of the said compound to be evaluated and said binding region in order to provide an energy-minimized configuration of the said compound in the binding region;
d) evaluating the results of said fitting operation to optimize the binding characteristics of said compound to an albumin binding site.

21. A method of producing a computer readable database comprising the three-dimensional molecular structural coordinates of one or more human albumin binding regions selected from the group consisting of the binding region IA, IA/IB, IA/IIA, IB, I/II; I/III; II/III, IIA, IIA/IIB, IIB, IIIA, IIIA/IIIB, IIIB and IIIB′, said method comprising a) obtaining three-dimensional structural coordinates defining said binding regions; and b) introducing said structural coordinates into a computer to produce a database containing the molecular structural coordinates of said binding regions.

22. A computer readable database produced by the method of claim 21.

23. The method of claim 21 further comprising utilizing the structural representations stored in said database for predictive ADME.

24. A method of producing a computer readable database comprising a representation of a compound capable of binding one or more human albumin binding subdomains, said method comprising a) introducing into a computer program a computer readable database produced by claim 1; b) generating a three-dimensional representation of one or more human albumin binding subdomains in said computer program; c) superimposing a three-dimensional model of at least one binding test compound on said representation of said one or more binding subdomains; d) assessing whether said test compound model fits spatially into one or more human serum albumin binding subdomains; and e) storing a structural representation of a compound that fits into one or more human serum albumin binding subdomains.

25. A method of producing a computer readable database comprising a representation of a compound capable of binding one or more human albumin binding subdomains, said method comprising a) introducing into a computer program a computer readable structural database comprising the three-dimensional molecular structural coordinates of one or more human albumin binding regions selected from the group consisting of the binding region IA, IA/IB, IA/IIA, IB, I/II; I/III; II/III, IIA, IIA/IIB, IIB, IIIA, IIIA/IIIB, IIIB and IIIB′, said computer readable structural database produced by a method comprising obtaining three-dimensional structural coordinates defining said binding regions and introducing said structural coordinates into a computer to produce a database containing the molecular structural coordinates of said binding regions; b) generating a three-dimensional representation of one or more human albumin binding subdomains in said computer program; c) superimposing a three-dimensional model of at least one binding test compound on said representation of said one or more binding subdomains; d) assessing whether said test compound model fits spatially into one or more human serum albumin binding subdomains; and e) storing a structural representation of a compound that fits into one or more human serum albumin binding subdomains.

26. A computer readable database produced by the method of claim 25.

27. The method of claim 25 further comprising utilizing the structural representations stored in said database for predictive ADME.

28. An isolated protein fragment comprising a human serum albumin binding subdomain selected from the group consisting of binding region IA, IA/IB, IA/IIA, IB, I/II; I/III; II/III, IIA/IIB, IIB, IIIA/IIIB, IIIB and IIIB′.

29. A method of determining the binding affinity of a drug to a target human serum albumin binding subdomain selected from the group consisting of human binding subdomain selected from the group consisting of binding region IA, IA/IIB, IA/IIA, IB, I/II; I/III; II/III, IIA/IIB, IIB, IIIA/IIIB, IIIB and IIIB′ comprising the steps of isolating a protein fragment according to claim 28, introducing said protein fragment to said drug in an amount and for a time sufficient to block the site on that drug that will bind to the target albumin binding subdomain, and then determining the level of human serum albumin binding of the drug following said introduction of said protein fragment in order to determine the binding affinity of the drug to the target albumin binding subdomain.

30. The method of claim 29 further comprising a step of using the determined binding affinity to the target binding subdomain to assess the likelihood that the drug will displace a molecule or compound at the target binding subdomain.

31. A kit for performing the method of claim 29 comprising an isolated protein fragment comprising a human serum albumin binding subdomain selected from the group consisting of binding region IA, IA/IB, IA/IIA, IB, I/II; I/III; II/III, IIA/IIB, IIB, IIIA/IIIB, IIIB and IIIB′ in an amount sufficient to block the site on a drug that would bind to a human serum albumin binding domain, a means to allow the introduction of the isolated fragment to a drug being assessed, and means to assess the binding of human serum albumin to the drug following introduction of the isolated fragment for a time sufficient to allow binding to take place.

32. A method of assessing the binding affinity of a drug to a target human serum albumin binding subdomain selected from the group consisting of binding subdomain selected from the group consisting of binding region IA, IA/IB, IA/IIA, IB, I/II; I/III; II/III, IIA/IIB, IIB, IIIA/IIIB, IIIB and IIIB′ comprising the steps of obtaining a human serum albumin having a target binding subdomain that is blocked, introducing said blocked albumin to said drug and then determining the level of binding of the drug to the human serum albumin with a blocked target binding subdomain in order to assess the binding affinity of the drug to the target albumin binding subdomain.

33. The method of claim 32 further comprising a step of using the determined binding affinity to the target binding subdomain to assess the likelihood that the drug will displace a molecule or compound at the target binding subdomain.

34. A kit for performing the method of claim 32 comprising human serum albumin having a target binding subdomain that is blocked, a means to allow the introduction of the blocked human serum albumin to a drug being assessed, and means to assess the binding of the blocked human serum albumin to the drug being assessed.
